SEQUENCE LISTING (10 sequences)

SEQ ID NO: 1 - protein, 5 aa, Homo sapiens
TYWLG

SEQ ID NO: 2 - protein, 17 aa, Homo sapiens
IMSPVDSDIRYSPSFQG

SEQ ID NO: 3 - protein, 10 aa, Homo sapiens
RRPGQGYFDF

SEQ ID NO: 4 - protein, 11 aa, Homo sapiens
RASQGISSWLA

SEQ ID NO: 5 - protein, 7 aa, Homo sapiens
AASSLQS

SEQ ID NO: 6 - protein, 9 aa, Homo sapiens
QQYNIYPYT

SEQ ID NO: 7 - protein, 119 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 108 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 9 - protein, 449 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0176330 A1
(43) Pub. Date: Jun. 25, 2026

(54) COMPOSITIONS OF ANTI-INTERLEUKIN 12/INTERLEUKIN 23 ANTIBODY

(71) Applicant: AMGEN INC., Thousand Oaks, CA (US)

(72) Inventors: Qian LIU, Thousand Oaks, CA (US); Juma BRIDGEWATER, Thousand Oaks, CA (US); Siddharth PRABHU, Thousand Oaks, CA (US); Joshua TOKUDA, Thousand Oaks, CA (US); Dong XIANG, Thousand Oaks, CA (US); Marisa JOUBERT, Thousand Oaks, CA (US); Alla POLOZOVA, Thousand Oaks, CA (US); Ahmed ELBARADEI, Thousand Oaks, CA (US)

(73) Assignee: AMGEN INC., Thousand Oaks, CA (US)

(21) Appl. No.: 19/124,318

(22) PCT Filed: Oct. 27, 2023
(86) PCT No.: PCT/US2023/078032

§ 371 Date: Apr. 25, 2025

Related U.S. Application Data
(60) Provisional application No. 63/381,459, filed on Oct. 28, 2022.
(60) Provisional application No. 63/583,438, filed on Sep. 18, 2023.
(60) Provisional application No. 63/591,990, filed on Oct. 20, 2023.

(51) Int. Cl. C07K 16/00

(52) U.S. Cl. CPC C07K 16/00; C07K 2317/76; C07K 2317/92

(57) ABSTRACT

Pharmaceutical compositions comprising anti-interleukin 12 (IL-12) interleukin 23 (IL-23) antibodies are described. The pharmaceutical compositions may comprise molecular attributes, such as oxidation of light chain W32, isoaspartic acid at heavy chain position D55; high molecular weight species such as dimers and/or oligomers, high mannose, sialylation, or acidic peak species. Method comprising administering the pharmaceutical compositions are described. Methods of manufacturing the pharmaceutical compositions are described.

### REFERENCE TO RELATED APPLICATIONS

The present application claims the benefit of U.S. Provisional Application No. 63/381,459, filed Oct. 28, 2022, and U.S. Provisional Application No. 63/583,438, filed Sep. 18, 2023, and U.S. Provisional Application No. 63/591,990, filed Oct. 20, 2023, each of which is hereby incorporated by reference in its entirety.

### SEQUENCE LISTING

The present application is being filed along with a Sequence Listing in electronic format. The Sequence Listing is provided as a file entitled 10296-WO01-SEC_seqlist.xml, created Oct. 26, 2023, which is 10,470 kb in size. The information in the electronic format of the Sequence Listing is incorporated herein by reference in its entirety.

### FIELD

The instant disclosure relates to molecular attributes of antibodies and compositions thereof.

### BACKGROUND

A therapeutic agent that can be used to treat inflammatory conditions is an anti-interleukin 12 (IL-12)/interleukin 23 (IL-23) antibody. One example of such a therapeutic agent is ustekinumab, which is marketed as STELARA® product (Janssen Biotech, Inc.).

Molecular attributes define the physicochemical properties of therapeutic biological molecules, and can therefore impact the drug safety and efficacy. The levels of attributes critical to the drug quality, or critical quality attributes (CQAs), are explicitly defined by the product purity specifications subject to extensive regulatory reviews. There is a need for identifying and producing anti-IL-12/IL-23 antibody pharmaceutical compositions having suitable CQA profiles so that clinically acceptable pharmaceutical compositions may be made available to patients.

### SUMMARY

Pharmaceutical compositions and methods are described herein. By way of example, pharmaceutical compositions, methods comprising administering the pharmaceutical composition, and methods of manufacturing the pharmaceutical composition are described in the following paragraphs.

A pharmaceutical composition anti-interleukin 12 (IL-12)/interleukin 23 (IL-23) antibodies is described. The antibodies may comprise a heavy chain comprising a CDRH1, CDRH2, and CDRH3, in which the amino acid sequence of the CDRH1, CDRH2 and CDRH3 is SEQ ID NO: 1, 2 and 3, respectively; and a light chain comprising a CDRL1, CDRL2, and CDRL3, in which the amino acid sequence of the CDRL1, CDRL2, and CDRL3 is SEQ ID NO: 4, 5 and 6, respectively.

For the pharmaceutical composition, at least one of:
(a) the light chain comprises W32 (EU numbering), wherein at least 1% and no more than 2% of the light chains of the composition comprise oxidized W32; and/or
(b) the heavy chain comprises position D55 (EU numbering), and wherein greater than 6% and no more than 14.3% of said antibodies comprise isoaspartic acid at heavy chain position 55, or greater than 6% and no more than 14%, or greater than 7% and no more than 14% of said antibodies comprise isoaspartic acid at heavy chain position 55; and/or
(c) at least 0.4% and no more than 5.0%, or at least 0.4% and no more than 1.0% of said antibodies of the pharmaceutical composition are comprised by high molecular weight species selected from dimers, oligomers, and dimers and oligomers; and/or
(d) at least 1.8% and no more than 6.4%, or at least 1.8% and no more than 3.7%, or greater than 1.8% and no more than 6.4%, or greater than 1.8% and no more than 3.7%, or at least 2.0% and no more than 6.4%, or at least 2.0% and no more than 3.7% of said antibodies of the pharmaceutical composition comprise high mannose; and/or
(e) at least 26.5% and no more than 58%, or at least 26.5% and no more than 29.5%, or at least 26.5% and no more than 29%, or at least 27% and no more than 58%, or at least 27% and no more than 29.5%, or at least 27% and no more than 29% of said antibodies of the pharmaceutical composition comprise sialylation; and/or
(f) the composition comprises a total of at least 28.0%, or at least 28.0% and no more than 60.3% acidic peak species, or at least 28.0% and no more than 58.3% acidic peak species, or at least 28% and no more than 55.0% acidic peak species as determined by cation exchange high performance liquid chromatography (CEX-HPLC); and/or
(g) the heavy chain comprises W33 (EU numbered), wherein at least 0.1% and no more than 0.8%, or at least 0.2% and no more than 0.8%, at least 0.1% and no more than 0.4%, or at least 0.2% and no more than 0.4% of the heavy chains of the composition comprise oxidized W33.

For any of the pharmaceutical compositions described herein (including any of those of any preceding or subsequent paragraphs), one or more of the following may apply:
(a) the light chain comprises W32 (EU numbering), in which at least 1% and no more than 2% of the light chains of the composition comprise oxidized W32; and/or
(b) the heavy chain comprises position D55 (EU numbering), and greater than 6%, and no more than 14% of said antibodies comprise isoaspartic acid at heavy chain position 55; or greater than 6%, and no more than 13% of said antibodies comprise isoaspartic acid at heavy chain position 55; and/or
(c) at least 0.4% and no more than 5.0%, or at least 0.5% and no more than 5.0% of said antibodies of the pharmaceutical composition are comprised by high molecular weight species selected from dimers, oligomers, and dimers and oligomers; and/or
(d) at least 1.8% and no more than 3.7%, or greater than 1.8% and no more than 3.7% of said antibodies of the pharmaceutical composition comprise high mannose; and/or
(e) at least 26.5% and no more than 29.5% of said antibodies of the pharmaceutical composition comprise sialylation; and/or
(f) the composition comprises a total of at least 28.0% and no more than 60.3% acidic peak species, or at least 28.0% and no more than 58.3% acidic peak species, or at least 28% and no more than 55.0% acidic peak species as determined by cation exchange high performance liquid chromatography (CEX-HPLC).

For any of the pharmaceutical compositions described herein (including any of those of any preceding or subsequent paragraphs), one or more of the following may apply:
(a) the light chain comprises W32 (EU numbering), wherein greater than 1% and no more than 2% of the light chains of the composition comprise oxidized W32; and/or
(b) greater than 6%, and no more than 14% of said antibodies comprise isoaspartic acid at heavy chain position 55; and/or
(c) at least 0.4% and no more than 0.9%, or at least 0.5% and no more than 0.9% of said antibodies of the pharmaceutical composition are comprised by high molecular weight species selected from dimers, oligomers, and dimers and oligomers; and/or
(d) at least 2.0% and no more than 3.7% of said antibodies of the pharmaceutical composition comprise high mannose; and/or
(e) at least 26.5% and no more than 28.9% of said antibodies of the pharmaceutical composition comprise sialylation; and/or
(f) the composition comprises a total of at least 46.0% and no more than 60.3% acidic peak species as determined by CEX-HPLC.

For any of the pharmaceutical compositions described herein (including any of those of any preceding or subsequent paragraphs), one or more of the following may apply:
(a) the light chain comprises W32 (EU numbering), wherein greater than 1% and no more than 2% of the light chains of the composition comprise oxidized W32; and/or
(b) greater than 6%, and no more than 13% of said antibodies comprise isoaspartic acid at heavy chain position 55; and/or
(c) at least 0.4% and no more than 0.8% of said antibodies of the pharmaceutical composition are comprised by high molecular weight species selected from dimers, oligomers, and dimers and oligomers; and/or
(d) at least 2.0% and no more than 3.7% of said antibodies of the pharmaceutical composition comprise high mannose; and/or
(e) at least 27% and no more than 28.9% of said antibodies of the pharmaceutical composition comprise sialylation; and/or
(f) the composition comprises a total of at least 46.0% and no more than 55.0% acidic peak species as determined by CEX-HPLC.

For some pharmaceutical compositions (a), about 1% of the light chains of the composition comprise oxidized W32. For any of the pharmaceutical compositions described herein (including any of those of any preceding or subsequent paragraphs), (c) greater than 0.5% and no more than 0.8% of said antibodies of the pharmaceutical composition may be comprised by high molecular weight species selected from dimers, oligomers, and dimers and oligomers.

For any of the pharmaceutical compositions described herein (including any of those of any preceding or subsequent paragraphs), two or more of the lettered items may apply, for example (a) and (b); or (a) and (c); or (a) and (e) or (a) and (e); or (a) and (f); or (b) and (c); or (b) and (e); or (b) and (e); or (b) and (f); or (c) and (d); or (c) and (e); or (e) and (f); or (d) and (e); or (d) and (f); or (e) and (f).

For any of the pharmaceutical compositions described herein (including any of those of any preceding or subsequent paragraphs), three or more of the lettered items may apply, for example (a) and (b) and (c); or (a) and (b) and (d); or (a) and (b) and (e); or (a) and (b) and (f); or (a) and (e) and (d); or (a) and (c) and (e); or (a) and (c) and (f); or (a) and (d) and (e); or (a) and (d) and (f); or (e) and (c) and (d); or (b) and (d) and (e); or (b) and (e) and (f); or (c) and (d) and (e); or (c) and (d) and (f); or (d) and (e) and (f).

For any of the pharmaceutical compositions described herein (including any of those of any preceding or subsequent paragraphs), (a), (b), (c), (d), (e), and (f) may apply.

For any of the pharmaceutical compositions described herein (including any of those of any preceding or subsequent paragraphs), (a) the oxidized W32 may be determined by reduced peptide mapping. For any of the pharmaceutical compositions described herein (including any of those of any preceding or subsequent paragraphs), (a) the oxidized W32 may be determined by reduced peptide mapping comprising:
denaturing one or more of the antibodies;
reducing the one or more antibodies;
S-alkylating the one or more antibodies;
digesting the one or more antibodies with trypsin, thereby producing peptides; and
subjecting said peptides to reverse phase ultra-high performance liquid chromatography (RP-UHPLC).

For any of the pharmaceutical compositions described herein (including any of those of any preceding or subsequent paragraphs), (a) the oxidized W32, and/or (b) isoaspartic acid at heavy chain position 55, and/or (g) the oxidized W33, is determined by reduced peptide mapping with mass spectrometry (MS), in which the reduced peptide mapping comprises:
diluting one or more of the antibodies to a concentration of 1 mg/mL in 7.5M Guanidine HCl (GdnHCl), 0.25M TRIS, 2 mM EDTA, pH 7.5 and denaturing said one or more antibodies therein at 27° C. for 30 minutes;
reducing the one or more antibodies with 3 mM dithiothreitol (DTT) at 27±2° C. for 30±5 minutes;
S-alkylating the one or more antibodies with 7 mM iodoacetic acid at 27±2° C. for 15±2 minutes;
desalting the one or more antibodies;
digesting the one or more antibodies with 0.03 μg/μL trypsin at 37±2° C. for 30±5 minutes, thereby producing peptides;
quenching the digestion in 0.1 M urea; and
subjecting the peptides to RP-UHPLC on a C18 column over a gradient of 100% mobile phase A+0% mobile phase B, to 5% mobile phase A+95% mobile phase B, wherein the mobile phase gradient is generated with 0% mobile phase B from 0 min to 2 mins, 17% to 23% mobile phase B from 36 mins to 76 mins, 40% mobile phase B from 124 mins to 126 mins, 95% mobile phase B from 126.1 mins to 133 mins; and 0% mobile phase B from 133.1 mins to 144 mins, and wherein mobile phase A comprises 0.05 mM methionine 0.1% TFA in water, and wherein mobile phase B comprises 0.05 mM methionine 0.1% TFA in acetonitrile.

For any of the pharmaceutical compositions described herein (including any of those of any preceding or subsequent paragraphs), (c) the percentages of high molecular weight species may be as determined by size exclusion ultra-high performance liquid chromatography (SE-UHPLC). For any of the pharmaceutical compositions described herein (including any of those of any preceding or subsequent paragraphs), (c) the percentages of high molecular weight species may be as determined by SE-UHPLC comprising a 4.6×150 mm column of 1.7 μm particle size, said column equilibrated with a mobile phase of 250 mM Sodium Chloride 100 mM Sodium Phosphate pH 6.8, and detection of absorbance at 280 nm of eluate of said column, said SE-UHPLC comprising a flow rate of 0.4 mL/min, a sample temperature of 8±5° C., and 60 μg sample in an injection volume of no more than 20 μL.

For any of the pharmaceutical compositions described herein (including any of those of any preceding or subsequent paragraphs), (d) the percentages of high mannose and/or sialylation may be as determined by Hydrophilic Interaction Liquid Chromatography (HILIC). For any of the pharmaceutical compositions described herein (including any of those of any preceding or subsequent paragraphs), (d) the percentages of high mannose and/or sialylation may be as determined by HILIC with in-line fluorescence detection comprising:
digesting one or more of the antibodies at a concentration of no more than 60 mg/mL in PNGase F at 37±2° C. for 120±12 minutes, thereby releasing glycans from said antibodies;
labeling said glycans with 2-aminobenzoic acid (2-AA) in a solution comprising 12.5 mg/mL 2-AA and 125 mM sodium cyanoborohydride and 37.5% (v/v) methanol; and
subjecting said labeled glycans to chromatography on a 2.1×150 mm, 1.7 m glycan column over a gradient of 20% mobile phase A 80% mobile phase B, to 80% mobile phase A 20% mobile phase B, wherein the gradient is generated with 20% mobile phase A+80% mobile phase B from 0 to 1 mins, 42% mobile phase A+58% mobile phase B at 110 mins, 80% mobile phase A+20% mobile phase B from 111 mins to 115 mins, and 20% mobile phase A+80% mobile phase B from 120 mins to 125 mins, wherein mobile phase A comprises 100 mM ammonium formate pH 3.0, and wherein mobile phase B comprises 100% acetonitrile.

For any of the pharmaceutical compositions described herein (including any of those of any preceding or subsequent paragraphs), (e) the percentages of acidic peak species may be as determined by CEX-HPLC. For any of the pharmaceutical compositions described herein (including any of those of any preceding or subsequent paragraphs), (e) the percentages of acidic peak species may be as determined by CEX-HPLC comprising:
applying the antibodies to a 4.6 mm×100 mm, 5 μm particle size cation exchange column and eluting along a salt gradient at 30° C. and pH 7.0, wherein 60 μg of the antibodies are applied to the cation exchange column in an injection volume of 60 μL;
wherein the salt gradient is generated with 5% mobile phase B from 0 min to 2 min, 5% to 12% mobile phase B from 2 min to 21 min, 12% mobile phase B from 21 min to 21.1 min, 12% to 100% mobile phase B from 21.1 min to 21.5 min; 100% mobile phase B from 21.5 min to 23.5 min, 100% to 5% mobile phase B from 23.5 min to 24 min, and to 5% mobile phase B at 24 min to 29 min, wherein a mobile phase A comprises 20 mM sodium phosphate at pH 7.0, and the mobile phase B comprises 20 mM sodium phosphate, 500 mM sodium chloride at pH 7.0, and
wherein the mobile phase is applied to the column at a flow rate of 0.8 mL/min.

For any of the pharmaceutical compositions described herein (including any of those of any preceding or subsequent paragraphs), the anti-IL-12/IL-23 antibodies may inhibit IL-23 signaling as determined in an in vitro assay comprising:
(a) determination of a presence or absence of energy transfer between an amplified luminescent proximity donor bead and acceptor bead, said donor bead comprising biotinylated interleukin-12 receptor β1 (IL-12-R) immobilized thereon, and said acceptor bead comprising recombinant human interleukin-23 comprising a His tag (IL-23-His) immobilized thereon, wherein binding of said antibodies to a p40 subunit of the IL-23-His prevents the IL-23-His from binding to biotinylated IL-12-R; or
(b) determination of a presence or absence of luminescence by an engineered cell in the presence of IL-23 and said antibodies, said engineered cell expressing IL-23R, IL-12Rβ1, IL-23R, and a Sis-inducible element (SIE)-response element (RE) luciferase gene (Luc2P), wherein said SIE-RE Luc2P is responsive to IL-23 induced STAT signaling, wherein binding of said antibodies to a p40 subunit of IL-23 prevents said IL-23 from binding IL-12Rβ1.
   By way of example, the anti-IL-12/IL-23 antibodies may inhibit IL-23 by greater than 66%, or by at least 86% or at least 88%, compared to a reference of said IL-12/IL-23 antibodies that does not comprise oxidized light chain W32, as determined by the in vitro assay comprising (a). By way of example, the anti-IL-12/IL-23 antibodies may inhibit IL-23 by greater than 54%, or by at least 89% or at least 94%, compared to a reference of said IL-12/IL-23 antibodies that does not comprise oxidized light chain W32, as determined by the in vitro assay comprising (b). By way of example, the anti-IL-12/IL-23 antibodies may inhibit IL-23 to a greater degree than an ustekinumab composition wherein 44% of ustekinumab comprise oxidized light chain W32, said inhibition of IL-23 as determined by the in vitro assay comprising (a) or (b).

For any of the pharmaceutical compositions described herein (including any of those of any preceding or subsequent paragraphs), (i) the anti-IL-12/IL-23 antibodies comprising up to 3.7% high mannose may exhibit a difference in clearance no greater than 1.6% compared to reference anti-IL-12/IL-23 antibodies comprising 0.7% high mannose; or (ii) the anti-IL-12/IL-23 antibodies comprising up to 19.3% high mannose may exhibit a difference in clearance no greater than 10% compared to reference anti-IL-12/IL-23 antibodies comprising 0.7% high mannose.

For any of the pharmaceutical compositions described herein (including any of those of any preceding or subsequent paragraphs), (i) the anti-IL-12/IL-23 antibodies comprising up to 3.7% high mannose may increase clearance of said antibodies by no more than 1.6%, as measured by area under curve in a pharmacokinetic model, compared to a reference anti-IL-12/IL-23 antibodies comprising 0.7% high mannose, or (ii) the anti-IL-12/IL-23 antibodies comprising up to 19.3% high mannose may increase clearance of said antibodies by no more than 10%, as measured by area under curve in a pharmacokinetic model, compared to reference anti-IL-12/IL-23 antibodies comprising 0.7% high mannose.

For any of the pharmaceutical compositions described herein (including any of those of any preceding or subsequent paragraphs), the anti-IL-12/IL-23 antibodies comprising up to 3.7% high mannose may increase clearance of said antibodies by no more than 1.6%, as measured by area under curve in a pharmacokinetic model, compared to the reference anti-IL-12/IL-23 antibodies comprising 0.7% high mannose.

For any of the pharmaceutical compositions described herein (including any of those of any preceding or subsequent paragraphs), the anti-IL-12/IL-23 antibodies have a half-life of at least 24 days. For any of the pharmaceutical compositions described herein (including any of those of any preceding or subsequent paragraphs), the half-life is as determined by quantification of free anti-IL-12/IL-23 antibodies in serum by an electrochemiluminescence (ECL) assay comprising a first anti-idiotypic antibody immobilized on a substrate and a second anti-idiotypic antibody for detection, for a dose of anti-IL-12/IL-23 antibodies of 90 mg, subcutaneous.

For any of the pharmaceutical compositions described herein (including any of those of any preceding or subsequent paragraphs), the antibodies may comprise the heavy chain variable region of SEQ ID NO: 7 and the light chain variable region of SEQ ID NO: 8. For any of the pharmaceutical compositions described herein (including any of those of any preceding or subsequent paragraphs), the antibodies may comprise the heavy chain of SEQ ID NO: 9 and the light of SEQ ID NO: 10. For any of the pharmaceutical compositions described herein (including any of those of any preceding or subsequent paragraphs), the antibodies may comprise or consist of ustekinumab.

For any of the pharmaceutical compositions described herein (including any of those of any preceding or subsequent paragraphs), the pharmaceutical composition is a unit dose comprising no more than 90 mg of the antibodies, such as 45 mg.

For any of the pharmaceutical compositions described herein (including any of those of any preceding or subsequent paragraphs), the pharmaceutical composition is a unit dose comprising 90 mg of the antibodies.

For any of the pharmaceutical compositions described herein (including any of those of any preceding or subsequent paragraphs), the pharmaceutical composition is a unit dose comprising 45 mg of the antibodies.

For any of the pharmaceutical compositions described herein (including any of those of any preceding or subsequent paragraphs), the pharmaceutical composition is for medical use.

For any of the pharmaceutical compositions described herein (including any of those of any preceding or subsequent paragraphs), the pharmaceutical composition is for use in treating an inflammatory condition. Examples of inflammatory conditions include psoriasis, psoriatic arthritis, and Crohn's disease. Examples of inflammatory conditions include psoriasis, psoriatic arthritis, Crohn's disease, and ulcerative colitis.

For any of the pharmaceutical compositions described herein (including any of those of any preceding or subsequent paragraphs), the anti-IL-12/IL-23 antibodies are administered at a dose of 45 mg, 90 mg, 130 mg, 260 mg, 390 mg, or 520 mg, or the pharmaceutical composition comprises a dose of 45 mg, 90 mg, 130 mg, 260 mg, 390 mg, or 520 mg of the anti-IL-12/IL-23 antibodies.

For any of the pharmaceutical compositions described herein (including any of those of any preceding or subsequent paragraphs), the anti-IL-12/IL-23 antibodies are administered at a dose of 45 mg, or the pharmaceutical composition comprises a dose of 45 mg.

For any of the pharmaceutical compositions described herein (including any of those of any preceding or subsequent paragraphs), the anti-IL-12/IL-23 antibodies are administered at a dose of 90 mg, or the pharmaceutical composition comprises a dose of 90 mg.

Also described herein is the use of any pharmaceutical composition described herein for the manufacture of a medicament for treating an inflammatory condition. Examples of inflammatory conditions include psoriasis, psoriatic arthritis, and Crohn's disease. Examples of inflammatory conditions include psoriasis, psoriatic arthritis, Crohn's disease, and ulcerative colitis.

Also described herein is a method comprising administering any pharmaceutical composition described herein to a subject in need of treatment by the anti-IL-12/IL-23 antibodies. For some methods, the subject has an inflammatory condition. Also described herein is a method comprising administering any pharmaceutical composition described herein to a subject who has an inflammatory condition. Examples of inflammatory conditions include psoriasis, psoriatic arthritis, and Crohn's disease. Examples of inflammatory conditions include psoriasis, psoriatic arthritis, Crohn's disease, and ulcerative colitis. For some methods, the anti-IL-12/IL-23 antibodies are administered at a dose of 45 mg, 90 mg, 130 mg, 260 mg, 390 mg, or 520 mg. By way of example, the subject may be a human.

Also described herein is a method of manufacturing any pharmaceutical composition described herein. The method may comprise providing a composition comprising the anti-IL-12/IL-23 antibodies.

The method may comprise determining at least one of:
(a) an amount that light chains of the composition comprise oxidized W32; and/or
(b) an amount of antibodies of the pharmaceutical composition that comprise isoaspartic acid at position 55 of the heavy chain (EU numbering); and/or
(c) an amount of antibodies of the pharmaceutical composition that are comprised by high molecular weight species selected from dimers, oligomers, and dimers and oligomers; and/or
(d) an amount of antibodies of the pharmaceutical composition that comprise high mannose; and/or
(e) an amount of antibodies of the pharmaceutical composition that comprise sialylation; and/or
(f) an amount of antibodies of the pharmaceutical composition that comprise acidic peak species; and/or
(g) an amount that heavy chains of the composition that comprise oxidized W33.
   The method may further comprise manufacturing the composition for pharmaceutical use when the composition comprises a range of molecular attributes (oxidized light chain W32, isoaspartic acid at heavy chain position 55, high molecular weight species, high mannose, sialylation, acidic peak species, and/or oxidized W33) for any of the compositions pharmaceutical described here. For example, the method may further comprise manufacturing the composition for pharmaceutical use when:
(a) at least 1% and no more than 2% of the light chains of the composition comprise oxidized W32; and/or
(b) greater than 6% and no more than 14.3%, or greater than 6% and no more than 14%, or greater than 7% and no more than 14% of said antibodies comprise isoaspartic acid at heavy chain position 55; and/or
(c) at least 0.4% and no more than 5.0%, or at least 0.4% and no more than 1.0% of said antibodies of the pharmaceutical composition are comprised by high molecular weight species selected from dimers, oligomers, and dimers and oligomers; and/or
(d) at least 1.8% and no more than 6.4%, or greater than 1.8% and no more than 6.4%, or at least 1.8% and no more than 3.7%, or greater than 1.8% and no more than 3.7% of said antibodies of the pharmaceutical composition comprise high mannose; and/or
(e) at least 26.5% and no more than 58%, or at least 26.5% and no more than 29.5%, or at least 26.5% and no more than 29%, or at least 27% and no more than 58%, or at least 27% and no more than 29.5%, or at least 27% and no more than 29% of said antibodies of the pharmaceutical composition comprise sialylation; and/or
(f) the composition comprises a total of at least 28.0% acidic peak species, or at least 28.0% and no more than 60.3% acidic peak species, or at least 28.0% and no more than 58.3% acidic peak species, or at least 28% and no more than 55.0% acidic peak species as determined by CEX-HPLC; and/or
(g) at least 0.1% and no more than 0.8%, or at least 0.2% and no more than 0.8%, at least 0.1% and no more than 0.4%, or at least 0.2% and no more than 0.4% of the heavy chains of the composition comprise oxidized W33.
   For any method of manufacturing any pharmaceutical composition described herein (including any of those of any preceding or subsequent paragraphs), the method may further comprise manufacturing the composition for pharmaceutical use when:
(a) at least 1% and no more than 2% of the light chains of the composition comprise oxidized W32; and/or
(b) greater than 6%, and no more than 13% of said antibodies comprise isoaspartic acid at heavy chain position 55 or greater than 6%, and no more than 14% of said antibodies comprise isoaspartic acid at heavy chain position 55; and/or
(c) at least 0.4% and no more than 5.0%, or at least 0.5% and no more than 5.0% of said antibodies of the pharmaceutical composition are comprised by high molecular weight species selected from dimers, oligomers, and dimers and oligomers; and/or
(d) greater than 1.8% and no more than 3.7% of said antibodies of the pharmaceutical composition comprise high mannose; and/or
(e) at least 26.5% and no more than 29.5% of said antibodies of the pharmaceutical composition comprise sialylation; and/or
(f) the composition comprises a total of at least 28.0% and no more than 55.0% acidic peak species or at least 28.0% and no more than 60.3% acidic peak species as determined by CEX-HPLC.

For some methods of manufacturing, the composition may be manufactured for pharmaceutical use when (a) greater than 1% and no more than 2% of the light chains of the composition comprise oxidized W32. For some methods of manufacturing, the composition may be manufactured for pharmaceutical use when about 1% of the light chains of the composition comprise oxidized W32.

### BRIEF DESCRIPTION OF DRAWINGS

FIGS. 1A-1B are a series of graphs showing forced degradation (FD) rate comparison of desialylated and sialylated Reference Product and ustekinumab compositions in accordance with some embodiments herein.

FIGS. 2A-2C are a series of graphs showing markers of T cell activation for desialylated and sialylated ustekinumab compositions in accordance with some embodiments herein.

FIGS. 3A-3B are a series of graphs showing markers of T cell activation for desialylated and sialylated ustekinumab compositions in accordance with some embodiments herein.

### DETAILED DESCRIPTION

Described herein are pharmaceutical compositions comprising anti-IL-12/IL-23 antibodies such as ustekinumab. Several molecular attributes of anti-IL-12/IL-23 antibodies (e.g., ustekinumab), including oxidation of the light chain at Trp32 (EU numbering; this residue may also be referred to as “W32”, with reference to SEQ ID NO: 8), isoaspartic acid at heavy chain position 55 (EU numbering, residue D55 with reference to SEQ ID NO: 7), high molecular weight species such as dimers and/or oligomers, high mannose, sialylation, and acidic peak species (which may be determined by cation exchange high performance liquid chromatography (CEX-HPLC)) can impact the potency and biochemical properties of the anti-IL-12/IL-23 antibodies. Reported herein is analysis of molecular attribute profiles of anti-IL-12/IL-23 antibodies in pharmaceutical composition and determined to have an acceptable clinical profile, including potency profile. Molecular attributes of anti-IL-12/IL-23 antibodies in pharmaceutical compositions were measured throughout product shelf life, including up to end-of-shelf life. Unexpectedly, it was determined that pharmaceutical formulations of anti-IL-12/IL-23 antibodies described herein can comprise molecular attribute ranges considerably broader than those that had conventionally been specified for an anti-IL-12/IL-23 antibodies (e.g., STELARA® product, which is a commercially available reference product that comprises ustekinumab), thus enhancing the options for efficiently manufacturing safe and effective pharmaceutical compositions comprising anti-IL-12/IL-23 antibodies such as ustekinumab, while minimizing waste. In some embodiments, pharmaceutical compositions comprising anti-IL-12/IL-23 antibodies are provided, in which the antibodies are ustekinumab, comprising ranges of molecular attributes as described herein.

It was observed herein that chemical oxidation of tryptophan in ustekinumab, such as oxidation of the light chain at position W32, results in a reduction of potency of the antibody, as measured in IL-12 and IL-23 cell-based reporter gene bioassays (Example 4). It was further observed that compositions comprising anti-IL-12/IL-23 antibodies such as ustekinumab in clinical studies could comprise light chain W32 oxidation of up to at least 1%, and up to least 2% and be suitable for medical use (Example 12). Accordingly, it will be appreciated that pharmaceutical compositions comprising anti-IL-12/IL-23 antibodies of embodiments herein may comprise specified ranges of tryptophan oxidation (e.g., light chain W32 oxidation of at least 1% to less than 2%) possess suitable potency for medical use (See Examples 2-3). On the other hand, at end-of-shelf life for an available ustekinumab Reference Product (STELARA® product), the latest timepoint that the Reference Product would be considered suitable for clinical use, W32 oxidation was observed to be less than 1% (Example 3), and Reference Product in clinical studies was observed to comprise up to 0.30% light change W32 oxidation (Example 12). The percent light chain W32 oxidation may be as determined by reduced peptide mapping with mass spectrometry (MS). Suitable reduced peptide mapping with MS methods are described herein. It was further observed that compositions comprising anti-IL-12/IL-23 antibodies such as ustekinumab in clinical studies could comprise heavy chain W33 oxidation of up to at least 0.40%, and up to least 0.80% and be suitable for medical use (Example 12). Reference Product in these studies comprised 0.05% heavy chain W33 oxidation. The percent heavy chain W33 oxidation may be as determined by reduced peptide mapping with mass spectrometry (MS). Suitable reduced peptide mapping with MS methods are described herein. Accordingly, it will be appreciated that pharmaceutical compositions comprising anti-IL-12/IL-23 antibodies of embodiments herein may comprise specified ranges of tryptophan oxidation of greater than 0.05% heavy chain W33 oxidation up to 0.8%, or up to 0.4%, and be suitable for medical use. In some embodiments, the pharmaceutical compositions greater than 0.05% up to 0.8%, or greater than 0.05% up to 0.4%, or 0.1% to 0.8%, or 0.1% to 0.4% of the anti-IL-12/IL-23 antibodies comprise heavy chain W33 oxidation.

Isomerization of aspartic acid is another molecular attribute that is contemplated to impact potency. The CDRs of canonical antibodies determine and participate in the interaction of antibodies with their antigen. It has been observed that the aspartic acid at ustekinumab heavy chain position 55 (which is located in HCDR1) is subject to isomerization. Available Reference Product at end-of-shelf life contained isoaspartic acid levels at heavy chain position 55 of up to 6%. However, it is shown herein that anti-IL-12/IL-23 pharmaceutical compositions comprising up to 13% isoaspartic acid at heavy chain position 55 (with reference to SEQ ID NO: 7), such as greater than 6% to no more than 13% are suitable for medical use (Examples 2-3 and 10), as are anti-IL-12/IL-23 pharmaceutical compositions comprising up to 14%, or up to 14.3% isoaspartic acid at heavy chain position 55 (with reference to SEQ ID NO: 7)(Examples 7 and 10). In some embodiments, the pharmaceutical compositions of anti-IL-12/IL-23 antibodies comprise greater than 6% and up to 12%, or 7% to 13%, or greater than 6% and up to 14%, or greater than 6% and no more than 14.3%, or 7% to 14%, or 7% to 14.3% isoaspartic acid at heavy chain position 55. The percent isoaspartic acid may be determined by reduced peptide mapping with mass spectrometry (MS). Suitable reduced peptide mapping with MS methods are described herein. It will be understood that references to isoaspartic acid at position D55 (EU numbering), along with similar wording, does not imply that there are both aspartic acid and isoaspartic acid at the same position of the same polypeptide, but rather will refer to the presence of the isomerized form of the aspartic acid that is typically in position 55 of the polypeptide.

Acidic peaks may comprise species such as sialylated species, fragmented and partially reduced species, heavy chain glycation species, and pyroglutamylated N-terminal variants. As such, acidic peak species can impact potency and stability of the antibody. In Reference Product at end of shelf life, acidic peaks were observed to range from 14.3-27.6% (Example 3). However, it is shown herein that ustekinumab pharmaceutical compositions suitable for medical use may comprise up to 55.0% acidic peak species, as determined by cation-exchange high performance liquid chromatography (CEX-HPLC) (Example 3), or up to 60.3% acidic peak species as determined by CEX-HPLC (Example 7), and may comprise up to 55.3% acidic peak species, as determined by CEX-HPLC (Example 13), and may comprise up to 110.6% acidic peak species, as determined by CEX-HPLC (Example 13). Moreover, a release specification of up to 58.3% acidic peak species was established for ustekinumab pharmaceutical compositions suitable for medical use described herein. In some embodiments, the pharmaceutical compositions comprising anti-IL-12/IL-23 antibodies comprise greater than 28.0% acidic peak species. The pharmaceutical composition may be for a 45 mg dose of anti-IL-12/IL-23 antibody. In some embodiments, the pharmaceutical compositions comprising anti-IL-12/IL-23 antibodies comprise greater than 28.0%, and up to 55.3% acidic peak species. The pharmaceutical composition may be for a 90 mg dose of anti-IL-12/IL-23 antibody. In some embodiments, the pharmaceutical compositions comprising anti-IL-12/IL-23 antibodies comprise greater than 28.0%, and up to 60.3% acidic peak species. In some embodiments, the pharmaceutical compositions comprising anti-IL-12/IL-23 antibodies comprise greater than 30.0% acidic peak species. The pharmaceutical composition may be for a 45 mg dose of anti-IL-12/IL-23 antibody. In some embodiments, the pharmaceutical compositions comprising anti-IL-12/IL-23 antibodies comprise greater than 30.0%, and up to 55.3% acidic peak species The pharmaceutical composition may be for a 90 mg dose of anti-IL-12/IL-23 antibody. In some embodiments, the pharmaceutical compositions comprising anti-IL-12/IL-23 antibodies comprise greater than 30.0%, and up to 60.3% acidic peak species. In some embodiments, the pharmaceutical compositions comprising anti-IL-12/IL-23 antibodies comprise greater than 28.0%, and up to 58.3% acidic peak species. In some embodiments, the pharmaceutical compositions comprising anti-IL-12/IL-23 antibodies comprise greater than 30.0%, and up to 58.3% acidic peak species. In some embodiments, the pharmaceutical compositions comprising anti-IL-12/IL-23 antibodies comprise greater than 28.0%, and up to 55.0% acidic peak species. The acidic peak species may be as determined by CEX-HPLC. Suitable CEX-HPLC methods are described herein.

High molecular weight species, such as oligomers and dimers can impact the potency and/or immunogenicity of some antibodies. Conventionally, STELARA® product considered suitable for clinical use was observed to contain 0.1-0.5% HMW species (Examples 2-3). It was observed that pharmaceutical compositions comprising anti-IL-12/IL-23 antibodies (such as ustekinumab) suitable for medical use may comprise up to 0.8% HMW species, or up to 0.9% HMW species (See Examples 2-3 and 7), and may comprise up to 0.5% HMW species (Example 9), and may comprise up to 1.0% HMW species (Example 9). Moreover, a release criterion of up to 5.0% HMW species has been considered to define suitable compositions of anti-IL-12/IL-23 antibodies (such as ustekinumab) for clinical use. In some embodiments, the pharmaceutical compositions comprising anti-IL-12/IL-23 antibodies comprise at least 0.4% and no more than 5.0% HMW species (or at least 0.4% and no more than 1.0% HMW species), such as oligomer and dimers. In some embodiments, the pharmaceutical compositions comprising anti-IL-12/IL-23 antibodies comprise greater than 0.5% and up to 5.0% HMW species (or at least 0.6% and up to 5.0% HMW species), such as oligomer and dimers. The HMW species may comprise or consist of or consist essentially of anti-IL-12/IL-23 antibodies. In some embodiments, the pharmaceutical compositions comprising anti-IL-12/IL-23 antibodies comprise greater than 0.5% and up to 0.8% HMW species (or at least 0.6% up to 0.8% HMW species), or greater than 0.5% and up to 0.9% HMW species (or at least 0.6% and up to 0.9% HMW species), such as oligomer and dimers. For any of the pharmaceutical compositions described herein, the HMW species may be determined by SE-HPLC. Suitable SE-HPLC methods are described herein.

Pharmaceutical compositions of anti-IL-12/IL-23 antibodies (such as ustekinumab) having high mannose levels of 19.3% are modeled to have 10% increased clearance compared to anti-IL-12/IL-23 antibody having high mannose levels of 0.7% (Example 5). Typically, an increase of clearance of no more than 10% compared to a reference molecule is considered a pharmaceutically comparable amount of clearance compared to the reference molecule. Pharmaceutical compositions comprising anti-IL-12/IL-23 antibodies (such as ustekinumab) having greater than 1.8% high mannose and no more than 3.7% high mannose are modeled to increase clearance by no more than 1.6%. Thus, while it is contemplated that the presence of high mannose can impact clearance of antibodies, pharmaceutical compositions comprising anti-IL-12/IL-23 antibody (such as ustekinumab) of some embodiments having high mannose levels of greater than 1.8% and no more than 3.7% have pharmacokinetic properties suitable for clinical use. High mannose levels may be determined by HILIC. Suitable HILIC methodologies are described herein. Pharmaceutical compositions comprising anti-IL-12/IL-23 antibody (such as ustekinumab) of some embodiments having high mannose levels of greater than 1.8% and no more than 19.3% have pharmacokinetic properties suitable for clinical use. It was further observed in connection with clinical studies that pharmaceutical compositions comprising anti-IL-12/IL-23 antibodies (such as ustekinumab) suitable for medical use may comprise up to 3.7% high mannose species (Example 11), and may comprise up to 6.4% high mannose species (Example 11). Reference Product in these clinical studies was observed to comprise up to 1.03% high mannose species (Example 11). In some pharmaceutical compositions described herein, at least 1.8% and no more than 6.4% of the anti-IL-12/IL-23 antibodies (such as ustekinumab) comprise high mannose. The pharmaceutical composition may be for a 45 mg dose of anti-IL-12/IL-23 antibody. In some pharmaceutical compositions described herein, at least 2.0% and no more than 6.4% of the anti-IL-12/IL-23 antibodies (such as ustekinumab) comprise high mannose. In some pharmaceutical compositions described herein, least 2.0% and no more than 3.7% of the anti-IL-12/IL-23 antibodies (such as ustekinumab) comprise high mannose. The pharmaceutical composition may be for a 90 mg dose of anti-IL-12/IL-23 antibody. The pharmaceutical composition may be for a 45 mg dose of anti-IL-12/IL-23 antibody. In some pharmaceutical compositions described herein, at least 1.8% and no more than 3.7% of the anti-IL-12/IL-23 antibodies (such as ustekinumab) comprise high mannose. The pharmaceutical composition may be for a 90 mg dose of anti-IL-12/IL-23 antibody.

Sialylation levels can potentially contribute to the anti-inflammatory mechanism of action of the molecule (See, e.g., Cobb, BA (2020). “The history of IgG glycosylation and where we are now.” Glycobiology 30(4): 202-213). Based on modeling of sialylation exposure in clinical trials of ustekinumab compositions, it was observed that assessed sialylation levels of up to 29.0% were not associated with adverse events (Example 6.1), and sialylation levels of up to 58.0% were not associated with adverse events (Example 6.2). Accordingly, it is contemplated that pharmaceutical compositions comprising anti-IL-12/IL-23 antibody (such as ustekinumab) having sialylation levels of up to at least 29.0%, such as at least 26.5% and no more than 58%, or at least 26.5% and no more than 29.5%, or at least 26.5% and no more than 29.0%, or greater than 26.5% and no more than 58%, or greater than 26.5% and no more than 29.5%, or greater than 26.5% and no more than 29.0%, or at least 27% and no more than 29.5%, or at least 27% and no more than 29%, or at least 27% and no more than 58% are suitable for medical use. Sialylation levels may be determined by HILIC. Suitable HILIC methodologies are described herein. In some pharmaceutical compositions described herein, at least 26.5% and no more than 58% of the anti-IL-12/IL-23 antibodies (such as ustekinumab) of the pharmaceutical composition comprise sialylation. The pharmaceutical composition may be for a 45 mg dose of anti-IL-12/IL-23 antibody. In some pharmaceutical compositions described herein, at least 27% and no more than 58% of the anti-IL-12/IL-23 antibodies (such as ustekinumab) of the pharmaceutical composition comprise sialylation. The pharmaceutical composition may be for a 45 mg dose of anti-IL-12/IL-23 antibody. In some pharmaceutical compositions described herein at least 26.5% and no more than 29.5% of the anti-IL-12/IL-23 antibodies (such as ustekinumab) of the pharmaceutical composition comprise sialylation. In some pharmaceutical compositions described herein at least 27% and no more than 29.5% of the anti-IL-12/IL-23 antibodies (such as ustekinumab) of the pharmaceutical composition comprise sialylation. In some pharmaceutical compositions described herein, at least 26.5% and no more than 29% of the anti-IL-12/IL-23 antibodies (such as ustekinumab) of the pharmaceutical composition comprise sialylation. The pharmaceutical composition may be for a 90 mg dose of anti-IL-12/IL-23 antibody.

### Anti-IL-12/IL-23 Antibody

Anti-IL-12/IL-23 antibodies are described herein, and may be used in any of the compositions or methods described herein. The anti-IL-12/IL-23 antibody may bind to IL-12 and IL-23. In particular, the anti-IL-12/IL-23 antibody may bind to the p40 subunit of IL-12 and IL-23. For example, the antibody may bind to a p40 subunit of IL-23, and prevent the IL-23 from binding IL-12R. Unless expressly stated otherwise, all amino acid numbering for antibodies described herein will refer to EU numbering.

As used herein, “antibody” has its customary and ordinary meaning as understood by one of ordinary skill in the art in view of this disclosure. It refers to an immunoglobulin with specific binding to the target antigen, and includes, for instance, humanized, fully human, and monoclonal antibodies. For example, human antibodies can be of any isotype, including IgG (including IgG1, IgG2, IgG3 and IgG4 subtypes), IgA (including IgA1 and IgA2 subtypes), IgM and IgE. A human IgG antibody generally will comprise two full-length heavy chains and two full-length light chains. It will be understood that once an antibody is obtained from a source, it may undergo further engineering, for example to enhance stability and folding. Accordingly, it will be understood that a “human” antibody may be obtained from a source, and may undergo further engineering. The engineered antibody may still be referred to as a type of human antibody. Similarly, engineered variants of a human antibody, for example those that have undergone affinity maturation, will also be understood to be “human antibodies” unless stated otherwise. In some embodiments, the antigen binding protein comprises, consists essentially of, or consists of a human antibody.

A “heavy chain” of an antibody includes a variable region (“VH”), and three constant regions: CH1, CH2, and CH3. Example heavy chain constant regions include human IgG1, IgG2, IgG3, and IgG4 constant regions. A “light chain” of an antibody includes a variable region (“VL”), and a constant region (“CL”). Human light chains include kappa chains and lambda chains. While nucleic acids encoding heavy chains and light chains of antibodies may include signal peptides for the purpose of secretion of these molecules, it will be appreciated that antibodies typically refer to mature molecules that do not comprise signal peptides.

It will further be appreciated that when compositions comprising antibodies are produced, some antibodies may contain truncations of one or more of their polypeptides. For example, the signal peptide at the N-terminus is cleaved at a specific site by signal peptidase during co-translational translocation of proteins across the ER membrane, so is not part of the mature protein sequence. However, the fidelity of this reaction is not 100%, and low level miscleavage has been observed in some antibody products. Such miscleavage has given rise to heterogeneity in the mature protein, resulting in either some level of elongation or truncation of the N-terminus of the mature protein chains (Ying H, and Liu H). Identification of an alternative signal peptide cleavage site of mouse monoclonal antibodies by mass spectrometry. Immunol Lett 111: 66-8 (2007).; Beck A, and Liu H. Macro- and micro-heterogeneity of natural and recombinant IgG antibodies. Antibodies 8 (1): 18 (2019)).

The antibody heavy chain of all four IgG subclasses (IgG1, IgG2, IgG3, and IgG4) displays heterogeneity at its C-terminus (Shah B et al., Heavy-chain C-terminal amidation in therapeutic IgG does not pose a safety concern. J Pharm Sci, in press (2022); Beck A, and Liu H. Macro- and micro-heterogeneity of natural and recombinant IgG antibodies. Antibodies 8 (1): 18 (2019)). Therapeutic IgG mAbs are known to contain three C-terminal variants in their heavy chains: 1) the unprocessed C-terminal lysine (K), 2) the processed C-terminal K, and 3) C-terminal amidation (Tsubaki M, et al. C-terminal modification of monoclonal antibody drugs: amidated species as a general product-related substance. International journal of biological macromolecules. 52:139-47 (2013)). All three C-terminal variants (unprocessed C-terminal K, processed C-terminal K, and C-terminal amidation) have been detected in endogenous IgG from myeloma patients and human plasma, suggesting that they are a common modification in vivo (Shah B et al., Heavy-chain C-terminal amidation in therapeutic IgG does not pose a safety concern. J Pharm Sci, in press (2022)). Accordingly, it will be appreciated that compositions comprising antibodies described herein may comprise antibodies having C- and/or N-terminal truncations and/or elongations in at least some of their polypeptides. For example, a composition comprising ustekinumab may comprise antibodies comprising C- and/or N-terminal truncations and/or elongations of the heavy chain of SEQ ID NO: 9 or the light chain of SEQ ID NO: 10.

The anti-IL-12/IL-23 antibodies described herein may comprise heavy chain CDRs having the amino acid sequence of TYWLG (SEQ ID NO: 1) for HCDR1, IMSPVDSDIRYSPSFQG (SEQ ID NO: 2) for HCDR2, RRPGQGYFDF (SEQ ID NO: 3) for HCDR3, RASQGISSWLA (SEQ ID NO: 4) for LCDR1, AASSLQS (SEQ ID NO: 5) for LCDR2, and QQYNIYPYT (SEQ ID NO: 6) for LCDR3. The anti-IL-12/IL-23 antibodies comprising these heavy chain and light chain CDRs may be IgG1 kappa antibodies.

In some embodiments, the anti-IL-12/IL-23 antibodies comprise the heavy chain variable region of (for reference, W33 and D55 are underlined):
EVQLVQSGAEVKKPGESLKISCKGSGYSFTTYWLGWVRQMPGKGLDWIGIMSPVDSDIRYSPSFQGQVTMS VDKSITTAYLQWNSLKASDTAMYYCARRRPGQGYFDFWGQGTLVTVSS (SEQ ID NO: 7)
   The anti-IL-12/IL-23 antibodies may further comprise the light chain variable region of (for reference, W32 is underlined):

(SEQ ID NO: 8)
DIQMTQSPSSLSASVGDRVTITCRASQGISSWLAWYQQKPEKAPKSLIY
AASSLQSGVPSRFSGSGSGTDFTLTISSLQPEDFATYYCQQYNIYPYTF
GQGTKLEIKR.

The anti-IL-12/IL-23 antibodies may comprise the heavy chain variable region of SEQ ID NO: 7 and the light chain variable region of SEQ ID NO: 8, and be IgG1 kappa antibodies.

In some embodiments, the IL-12/IL-23 antibodies comprise the heavy chain of:

(SEQ ID NO: 9)
EVQLVQSGAEVKKPGESLKISCKGSGYSFTTYWLGWVRQMPGKGLDWIG
IMSPVDSDIRYSPSFQGQVTMSVDKSITTAYLQWNSLKASDTAMYYCAR
RRPGQGYFDFWGQGTLVTVSSSSTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK.

The anti-IL-12/IL-23 antibodies may further comprise the light chain of:

(SEQ ID NO: 10)
DIQMTQSPSSLSASVGDRVTITCRASQGISSWLAWYQQKPEKAPKSLIY
AASSLQSGVPSRFSGSGSGTDFTLTISSLQPEDFATYYCQQYNIYPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

Ustekinumab may comprise the heavy chain of SEQ ID NO: 9 and the light chain of SEQ ID NO: 10.

Any of the anti-IL-12/IL-23 antibodies described herein may be in a composition, such as a pharmaceutical composition. The pharmaceutical composition may comprise formulation materials for modifying, maintaining or preserving, for example, the pH, osmolality, viscosity, clarity, color, isotonicity, odor, sterility, stability, rate of dissolution or release, adsorption or penetration of the composition.

In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextran); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In some embodiments, the anti-IL-12/IL-23 antibodies are in a pharmaceutical composition comprising the anti-IL-12/23 antibodies L-histidine and L-histidine monohydrochloride monohydrate, polysorbate 80, and sucrose, at pH 5.7-6.3. For example, the pharmaceutical composition may comprise anti-IL-12/IL-23 antibodies (e.g., ustekinumab) at 90 mg/mL, L-histidine and L-histidine monohydrochloride monohydrate at 1 mg/mL, polysorbate 80 at 0.04 mg/mL, and sucrose at 76 mg/mL, at a pH of 5.7-6.3. For example, the pharmaceutical composition may comprise anti-IL-12/IL-23 antibodies (e.g., ustekinumab) at 5 mg/mL, L-histidine and L-histidine monohydrochloride monohydrate at 10 mM, sucrose at 8.5% (w/v), EDTA at 0.05 mM, L-methionine at 2.7 mM, polysorbate 80 at 0.040% (w/v), at a pH of 6.0.

### Molecular Attributes

Described herein are molecular attributes of anti-IL-12/IL-23 antibodies. “Molecular attributes” and variations of this root term has its ordinary and customary meaning as would be understood by one of ordinary skill in the art in view of this disclosure. It refers to a chemically or physically changed structure on a macromolecule, such as an antibody, and may be characterized in terms of its physicochemical identity or attribute type and location within the sequence of the macromolecule, e.g., the position of the amino acid on which the attribute is present. For example, tryptophan residues are susceptible to oxidation. For example, aspartic acid residues are subject to isomerization. For example, antibodies may comprise high mannose glycans, and/or sialylated glycans. For example, antibodies may be comprised in high molecular weight (HMW) species such as oligomers and dimers. For example, acidic peak species of an antibody may be a molecular attribute. Examples of acidic peak species include sialylated species, fragmented and partially reduced species, heavy chain glycation species, and pyroglutamylated N-terminal variants.

Exemplary molecular attribute types are described herein. For conciseness, molecular attributes may be referred to herein simply as “attributes.” The levels of attributes critical to the drug quality, or critical quality attributes (CQAs), may be explicitly defined by the product purity specifications. These specifications are typically subject to extensive regulatory reviews. In some embodiments, a specification may set the permissible levels of one or more molecular attributes in the manufacture of a biological therapy.

Examples of molecular attributes in pharmaceutical compositions comprising anti-IL-12/IL-23 antibodies described herein include tryptophan oxidation, such as oxidation of light chain W32 (EU numbering); oxidation of heavy chain W33 (EU numbering); isoaspartic acid at heavy chain position 55 (EU numbering); high molecular weight species selected from dimers and/or oligomers; high mannose; sialylation; and acidic peak species (it will be understood that acidic peak species may comprise on or more different molecular attributes as described herein). In some embodiments, a pharmaceutical composition comprising anti-IL-12/IL-23 antibodies may comprise a specified range of one or more of the listed molecular attributes. Examples of ranges of molecular attributes of anti-IL-12/IL-23 antibodies in pharmaceutical compositions suitable for medical use include those of the ustekinumab compositions shown in Tables 1, 2, and 7. In some embodiments, a pharmaceutical composition comprises anti-IL-12/IL-23 antibodies, having a molecular attribute range comprising a molecular attribute range or portion thereof of an ustekinumab composition in Table 1, 2 or 3, or 7 and which does not overlap with any Reference Product range of that molecular attribute in any of Tables 1, 2,3, and 7. The molecular attribute may include, for example, oxidation of light chain W32; isoaspartic acid at heavy chain position D55; high molecular weight species selected from dimers and/or oligomers; high mannose; sialylation, or acidic peak species. The composition may comprise two or more of the listed molecular attributes.

For pharmaceutical compositions and methods described herein, molecular attributes such as oxidation of light chain W32, oxidation of heavy chain W33; and isoaspartic acid at heavy chain position 55 may be determined by reduced peptide mapping with mass spectrometry (MS). The reduced peptide mapping with MS can comprise diluting one or more of the antibodies to a concentration of 1 mg/mL in 7.5M Guanidine HCl (GdnHCl), 0.25M TRIS, 2 mM EDTA, pH 7.5; reducing the one or more antibodies with 3 mM dithiothreitol (DTT) at 27±2° C. for 30 minutes; S-alkylating the one or more antibodies with 7 mM iodoacetic acid at 27±2° C. for 15±2 minutes; desalting the one or more antibodies, for example using a NAP-5 column; digesting the one or more antibodies with 0.03 μg/μL trypsin at 37±2° C. for 30±5 minutes, thereby producing peptides; quenching the digestion in 0.1 M urea; and subjecting the peptides to RP-UHPLC on a C18 column over a gradient of 100% mobile phase A+0% mobile phase B, to 5% mobile phase A+95% mobile phase B. For the reduced peptide mapping, the mobile phase A comprises 0.05 mM methionine 0.1% TFA in water, and the mobile phase B comprises 0.05 mM methionine 0.1% TFA in acetonitrile. The mobile phase gradient may be generated with 0% mobile phase B from 0 min to 2 mins, 17% to 23% mobile phase B from 36 mins to 76 mins, 40% mobile phase B from 124 mins to 126 mins, 95% mobile phase B from 126.1 mins to 133 mins; and 0% mobile phase B from 133.1 mins to 144 mins. After the peptides have been subjected to RP-UHPLC, the mass spectrum of the peptides may be obtained. By way of example, a suitable mass spectrometry instrument is a Thermo Scientific Q Exactive Orbitrap Mass Spectrometer.

For pharmaceutical compositions and methods described herein, molecular attributes such as high molecular weight species may be determined by size exclusion ultra-high performance liquid chromatography (SE-UHPLC). The SE-UHPLC may comprise a 4.6×150 mm column of 1.7 μm particle size, said column equilibrated with a mobile phase of 250 mM Sodium Chloride 100 mM Sodium Phosphate pH 6.8, and detection of absorbance at 280 nm of eluate of said column. The SE-UHPLC may further comprise a flow rate of 0.4 mL/min, a sample temperature of 8±5° C., and 60 μg sample in an injection volume of no more than 20 μL.

For pharmaceutical compositions and methods described herein, molecular attributes such as percentages of high mannose and/or sialylation are as determined by Hydrophilic Interaction Liquid Chromatography (HILIC). The HILIC may be HILIC with in-line fluorescence detection comprising digesting one or more of the antibodies at a concentration of no more than 60 mg/mL in PNGase F at 37±2° C. for 120±12 minutes, thereby releasing glycans from said antibodies. The HILIC may further comprise labeling the glycans with 2-aminobenzoic acid (2-AA) in a solution comprising 12.5 mg/mL 2-AA and 125 mM sodium cyanoborohydride and 37.5% (v/v) methanol. The HILIC may further comprise subjecting the labeled glycans to chromatography on a 2.1×150 mm, 1.7 μm glycan column over a gradient of 20% mobile phase A+80% mobile phase B, to 80% mobile phase A+20% mobile phase B. The gradient may be generated with 20% mobile phase A+80% mobile phase B from 0 to 1 mins, 42% mobile phase A+58% mobile phase B at 110 mins, 80% mobile phase A+20% mobile phase B from 111 mins to 115 mins, and 20% mobile phase A+80% mobile phase B from 120 mins to 125 mins. For the HILIC, the mobile phase A comprises 100 mM ammonium formate pH 3.0, and the mobile phase B comprises 100% acetonitrile.

For pharmaceutical compositions and methods described herein, molecular attributes such as percentages of acidic peak species may be determined by CEX-HPLC. The CEX-HPLC may comprise applying the antibodies to a 4.6 mm×100 mm, 5 μm particle size cation exchange column and eluting along a salt gradient at 30±2° C. and pH 7.0, wherein 60 μg of the antibodies are applied to the cation exchange column in an injection volume of 60 μL. The salt gradient of the CEX-HPLC may be generated with 5% mobile phase B from 0 min to 2 min, 5% to 12% mobile phase B from 2 min to 21 min, 12% mobile phase B from 21 min to 21.1 min, 12% to 100% mobile phase B from 21.1 min to 21.5 min; 100% mobile phase B from 21.5 min to 23.5 min, 100% to 5% mobile phase B from 23.5 min to 24 min, and to 5% mobile phase B at 24 min to 29 min. For the CEX-HPLX, the mobile phase A comprises 20 mM sodium phosphate at pH 7.0, and the mobile phase B comprises 20 mM sodium phosphate, 500 mM sodium chloride at pH 7.0. The mobile phase is applied to the column at a flow rate of 0.8 mL/min.

For pharmaceutical compositions and methods described herein, potency of the anti-IL-12/IL-23 antibodies may be determined based on inhibition of IL-23 signaling. Inhibition of IL-23 signaling may be determined by an in vitro assay. The in vitro assay may comprise
(a) determination of a presence or absence of energy transfer between an amplified luminescent proximity donor bead and acceptor bead, said donor bead comprising biotinylated interleukin-12 receptor β1 (IL-12-R) immobilized thereon, and said acceptor bead comprising recombinant human interleukin-23 comprising a His tag (IL-23-His) immobilized thereon, wherein binding of said antibodies to a p40 subunit of the IL-23-His prevents the IL-23-His from binding to biotinylated IL-12-R; or
(b) determination of a presence or absence of luminescence by an engineered cell in the presence of IL-23 and said antibodies, said engineered cell expressing IL-23R, IL-12Rβ1, IL-23R, and a Sis-inducible element (SIE)-response element (RE) luciferase gene (Luc2P), wherein said SIE-RE Luc2P is responsive to IL-23 induced STAT signaling, wherein binding of said antibodies to a p40 subunit of IL-23 prevents said IL-23 from binding IL-12Rβ1.

For some pharmaceutical compositions and methods described herein, the in vitro assay comprises (b), determination of a presence or absence of luminescence by an engineered cell in the presence of IL-23 and the antibodies.

### Pharmaceutical Compositions

Pharmaceutical compositions comprising anti-IL-12/IL-23 antibodies such as ustekinumab are described herein. The pharmaceutical composition may comprise anti-IL-12/IL-23 antibodies each comprising a heavy chain comprising a CDRH1, CDRH2, and CDRH3, in which the amino acid sequence of the CDRH1, CDRH2 and CDRH3 is SEQ ID NO: 1, 2 and 3, respectively, and a light chain comprising a CDRL1, CDRL2, and CDRL3, in which the amino acid sequence of the CDRL1, CDRL2, and CDRL3 is SEQ ID NO: 4, 5 and 6, respectively. For this pharmaceutical composition, at least one of:
(a) the light chain comprises W32 (EU numbering), wherein at least 1% and no more than 2% of the light chains of the composition comprise oxidized W32; and/or
(b) the heavy chain comprises position D55 (EU numbering), and wherein greater than 6% and no more than 14.3% of said antibodies comprise isoaspartic acid at heavy chain position 55, or greater than 6% and no more than 14%, or greater than 7% and no more than 14% of said antibodies comprise isoaspartic acid at heavy chain position 55; and/or
(c) at least 0.4% and no more than 5.0%, or at least 0.4% and no more than 1.0% of said antibodies of the pharmaceutical composition are comprised by high molecular weight species selected from dimers, oligomers, and dimers and oligomers; and/or
(d) at least 1.8% and no more than 6.4%, or greater than 1.8% and no more than 6.4%, or at least 1.8% and no more than 3.7%, or greater than 1.8% and no more than 3.7% of said antibodies of the pharmaceutical composition comprise high mannose; and/or
(e) at least 26.5% and no more than 58%, or at least 26.5% and no more than 29.5%, or at least 26.5% and no more than 29% of said antibodies of the pharmaceutical composition comprise sialylation; and/or
(f) the composition comprises a total of at least 28.0%, or at least 28.0% and no more than 60.3% acidic peak species, or at least 28.0% and no more than 58.3% acidic peak species, or at least 28% and no more than 55.0% acidic peak species as determined by cation exchange high performance liquid chromatography (CEX-HPLC); and/or
(g) the heavy chain comprises W33 (EU numbered 0, wherein at least 0.1% and no more than 0.8%, or at least 0.2% and no more than 0.8%, at least 0.1% and no more than 0.4%, or at least 0.2% and no more than 0.4% of the heavy chains of the composition comprise oxidized W33.

For some pharmaceutical compositions, at least one of:
(a) the light chain comprises W32 (EU numbering), wherein at least 1% and no more than 2% of the light chains of the composition comprise oxidized W32; and/or
(b) the heavy chain comprises position D55 (EU numbering), and wherein greater than 6.0%, and no more than 12.9% of said antibodies comprise isoaspartic acid at heavy chain position 55; and/or
(c) at least 0.4% and no more than 5.0%, or at least 0.5% and no more than 5.0% of said antibodies of the pharmaceutical composition are comprised by high molecular weight species selected from dimers, oligomers, and dimers and oligomers; and/or
(d) at least 1.8% and no more than 3.7%, or greater than 1.8% and no more than 3.7% of said antibodies of the pharmaceutical composition comprise high mannose; and/or
(e) at least 26.5% and no more than 29.5% of said antibodies of the pharmaceutical composition comprise sialylation; and/or
(f) the composition comprises a total of at least 28.0% and no more than 58.3% acidic peak species, or at least 28.0% and no more than 55.0% acidic peak species, as determined by cation exchange ultra-high performance liquid chromatography (CEX-HPLC).

Any of the pharmaceutical compositions described herein may comprise two or more of (a), (b), (c), (d), (e), and (f). For example, the pharmaceutical compositions described herein may comprise (a) and (b); or (a) and (c); or (a) and (d); or (a) and (e); or (a) and (f); or (b) and (c); or (b) and (d); or (b) and (e); or (b) and (f); or (c) and (d); or (c) and (e); or (c) and (f); or (d) and (e); or (d) and (f); or (e) and (f). Any of the pharmaceutical compositions described herein may comprise three or more of (a), (b), (c), (d), (e), and (f). For example, the pharmaceutical compositions described herein may comprise (a) and (b) and (c); or (a) and (b) and (d); or (a) and (b) and (e); or (a) and (b) and (f); or (a) and (c) and (d); or (a) and (c) and (e); or (a) and (c) and (f); or (a) and (d) and (e); or (a) and (d) and (f); or (b) and (c) and (d); or (b) and (d) and (e); or (b) and (e) and (f); or (c) and (d) and (e); or (c) and (d) and (f); or (d) and (e) and (f).

In some aspects, for the pharmaceutical composition, at least one of:
(a) the light chain comprises W32 (EU numbering), wherein at least 1% and no more than 2% of the light chains of the composition comprise oxidized W32; and/or
(b) the heavy chain comprises position D55 (EU numbering), and wherein greater than 6.0%, and no more than 14% of said antibodies comprise isoaspartic acid at heavy chain position 55; and/or
(c) at least 0.4% and no more than 5.0% of said antibodies of the pharmaceutical composition are comprised by high molecular weight species selected from dimers, oligomers, and dimers and oligomers; and/or
(d) at least 1.8% and no more than 3.7%, or greater than 1.8% and no more than 3.7% of said antibodies of the pharmaceutical composition comprise high mannose; and/or
(e) at least 26.5% and no more than 29.5% of said antibodies of the pharmaceutical composition comprise sialylation; and/or
(f) the composition comprises a total of at least 28.0% and no more than 60.3% acidic peak species, as determined by cation exchange ultra-high performance liquid chromatography (CEX-HPLC).
   Any of the pharmaceutical compositions described herein may comprise two or more of (a), (b), (c), (d), (e), and (f). For example, the pharmaceutical compositions described herein may comprise (a) and (b); or (a) and (c); or (a) and (d); or (a) and (e); or (a) and (f); or (b) and (c); or (b) and (d); or (b) and (e); or (b) and (f); or (c) and (d); or (c) and (e); or (c) and (f); or (d) and (e); or (d) and (f); or (e) and (f). Any of the pharmaceutical compositions described herein may comprise three or more of (a), (b), (c), (d), (e), and (f). For example, the pharmaceutical compositions described herein may comprise (a) and (b) and (c); or (a) and (b) and (d); or (a) and (b) and (e); or (a) and (b) and (f); or (a) and (c) and (d); or (a) and (c) and (e); or (a) and (c) and (f); or (a) and (d) and (e); or (a) and (d) and (f); or (b) and (c) and (d); or (b) and (d) and (e); or (b) and (e) and (f); or (c) and (d) and (e); or (c) and (d) and (f); or (d) and (e) and (f).
   For some pharmaceutical compositions, at least one of:
(a) the light chain comprises W32 (EU numbering), wherein greater than 1% and no more than 2% of the light chains of the composition comprise oxidized W32; and/or
(b) greater than 6.0%, and no more than 12.8% of said antibodies comprise isoaspartic acid at heavy chain position 55; and/or
(c) at least 0.4% and no more than 0.8% of said antibodies of the pharmaceutical composition are comprised by high molecular weight species selected from dimers, oligomers, and dimers and oligomers; and/or
(d) at least 2.0% and no more than 3.7% of said antibodies of the pharmaceutical composition comprise high mannose; and/or
(e) at least 27.0% and no more than 28.9% of said antibodies of the pharmaceutical composition comprise sialylation; and/or
(f) the composition comprises a total of at least 28.0% and no more than 55.0% acidic peak species as determined by CEX-HPLC, or at least 46.0% and no more than 55.0% acidic peak species.
   Any of the pharmaceutical compositions described herein may comprise two or more of (a), (b), (c), (d), (e), and (f). For example, the pharmaceutical compositions described herein may comprise (a) and (b); or (a) and (c); or (a) and (d); or (a) and (e); or (a) and (f); or (b) and (c); or (b) and (d); or (b) and (e); or (b) and (f); or (c) and (d); or (c) and (e); or (c) and (f); or (d) and (e); or (d) and (f); or (e) and (f). Any of the pharmaceutical compositions described herein may comprise three or more of (a), (b), (c), (d), (e), and (f). For example, the pharmaceutical compositions described herein may comprise (a) and (b) and (c); or (a) and (b) and (d); or (a) and (b) and (e); or (a) and (b) and (f); or (a) and (c) and (d); or (a) and (c) and (e); or (a) and (c) and (f); or (a) and (d) and (e); or (a) and (d) and (f); or (b) and (c) and (d); or (b) and (d) and (e); or (b) and (e) and (f); or (c) and (d) and (e); or (c) and (d) and (f); or (d) and (e) and (f).

For some pharmaceutical compositions, (c) at least 0.4% and no more than 5.0%, or at least 0.4% and no more than 1.0%, or greater than 0.5% and no more than 0.8% of the antibodies of the pharmaceutical composition are comprised by high molecular weight species selected from dimers and/or oligomers.

For any of the pharmaceutical composition described herein, (a) the oxidized W32 may be determined by reduced peptide mapping. By way of example, the reduced peptide mapping may comprise denaturing one or more of the antibodies; reducing the one or more antibodies; S-alkylating the one or more antibodies; digesting the one or more antibodies with trypsin, thereby producing peptides; and subjecting said peptides to reverse phase ultra-high performance liquid chromatography (RP-UHPLC). The mass spectrum of peptides subject to RP-UHPLC may then be obtained.

For any of the pharmaceutical composition described herein, (c) the percentages of high molecular weight species may be as determined by SE-UHPLC.

For any of the pharmaceutical composition described herein, the (d) the percentages of high mannose and/or sialylation may be as determined by HILIC.

For any of the pharmaceutical composition described herein, the (e) percentages of acidic peak species may be as determined by CEX-HPLC.

For any of the pharmaceutical composition described herein, the anti-IL12/IL-23 antibodies inhibit IL-23 signaling as determined in an in vitro assay comprising: (a) determination of a presence or absence of energy transfer between an amplified luminescent proximity donor bead and acceptor bead, said donor bead comprising biotinylated interleukin-12 receptor 131 (IL-12-R) immobilized thereon, and said acceptor bead comprising recombinant human interleukin-23 comprising a His tag (IL-23-His) immobilized thereon, wherein binding of said antibodies to a p40 subunit of the IL-23-His prevents the IL-23-His from binding to biotinylated IL-12-R; or (b) determination of a presence or absence of luminescence by an engineered cell in the presence of IL-23 and said antibodies, said engineered cell expressing IL-23R, IL-12Rβ1, IL-23R, and a Sis-inducible element (SIE)-response element (RE) luciferase gene (Luc2P), wherein said SIE-RE Luc2P is responsive to IL-23 induced STAT signaling, wherein binding of said antibodies to a p40 subunit of IL-23 prevents said IL-23 from binding IL-12Rβ1. It will be appreciated that the potency of any of the anti-IL12/IL-23 antibodies of any pharmaceutical composition described herein may be determined by such an in vitro assay.

By way of example, for some pharmaceutical compositions, the anti-IL-12/IL-23 antibodies inhibit IL-23 by greater than 66%, or by at least 86% or at least 88%, compared to a reference of said IL-12/IL-23 antibodies that does not comprise oxidized light chain W32, as determined by the in vitro assay comprising (a). By way of example, some pharmaceutical compositions, the anti-IL-12/IL-23 antibodies inhibit IL-23 to a greater degree than an ustekinumab composition in which 44% of ustekinumab comprise oxidized light chain W32, in which the inhibition of IL-23 is as determined by the in vitro assay comprising (a).

By way of example, for some pharmaceutical compositions, for some pharmaceutical compositions, the anti-IL-12/IL-23 antibodies inhibit IL-23 by greater than 54%, or by at least 89% or at least 94%, compared to a reference of said IL-12/IL-23 antibodies that does not comprise oxidized light chain W32, as determined by the in vitro assay comprising (b).

It will further be appreciated that for pharmaceutical compositions described herein, the anti-IL-12/IL-23 antibodies may have a suitable clearance for medical use, and as such, may have a specified range of high mannose. For example, for some pharmaceutical compositions, the anti-IL-12/IL-23 antibodies comprise up to 3.7% high mannose, and exhibit a difference in clearance no greater than 1.6% compared to reference anti-IL-12/IL-23 antibodies comprising 0.7% high mannose; or the anti-IL-12/IL-23 antibodies comprise up to 19.3% high mannose and exhibit a difference in clearance no greater than 10% compared to reference anti-IL-12/IL-23 antibodies comprising 0.7% high mannose. For some pharmaceutical compositions, the anti-IL-12/IL-23 antibodies comprising up to 3.7% high mannose exhibit an increase in clearance of the antibodies by no more than 1.6%, as measured by area under curve in a pharmacokinetic model, compared to a reference anti-IL-12/IL-23 antibodies comprising 0.7% high mannose, or the anti-IL-12/IL-23 antibodies comprising up to 19.3% high mannose exhibit increased clearance of the antibodies by no more than 10%, as measured by area under curve in a pharmacokinetic model, compared to reference anti-IL-12/IL-23 antibodies comprising 0.7% high mannose. For some pharmaceutical compositions, the anti-IL-12/IL-23 antibodies comprising up to 3.7% high mannose exhibit increased clearance of said antibodies by no more than 1.6%, as measured by area under curve in a pharmacokinetic model, compared to the reference anti-IL-12/IL-23 antibodies comprising 0.7% high mannose.

For some pharmaceutical compositions, at least 1.8% and no more than 6.4% of the anti-IL-12/IL-23 antibodies of the pharmaceutical composition comprise high mannose. For some pharmaceutical compositions, greater than 1.8% and no more than 6.4% of the anti-IL-12/IL-23 antibodies of the pharmaceutical composition comprise high mannose. For some pharmaceutical compositions at least 1.8% and no more than 3.7% of the anti-IL-12/IL-23 antibodies of the pharmaceutical composition comprise high mannose. For some pharmaceutical compositions greater than 1.8% and no more than 3.7% of the anti-IL-12/IL-23 antibodies of the pharmaceutical composition comprise high mannose. For some pharmaceutical compositions, at least 2.0% and no more than 6.4% of the anti-IL-12/IL-23 antibodies of the pharmaceutical composition comprise high mannose. For some pharmaceutical compositions at least 2.0% and no more than 3.7% of the anti-IL-12/IL-23 antibodies of the pharmaceutical composition comprise high mannose.

For any of the pharmaceutical compositions described herein, the anti-IL-12/IL-23 antibodies may have a half-life of at least 24 days. The half-life for any of the anti-IL-12/IL-23 antibodies described herein may be as determined by quantification of free anti-IL-12/IL-23 antibodies in serum by an electrochemiluminescence (ECL) assay comprising a first anti-idiotypic antibody immobilized on a substrate and a second anti-idiotypic antibody for detection, for a dose of anti-IL-12/IL-23 antibodies of 90 mg, subcutaneous.

By way of example, the anti-IL-12/IL-23 antibodies of the composition may comprise the heavy chain variable region of SEQ ID NO: 7 and the light chain variable region of SEQ ID NO: 8. The anti-IL-12/IL-23 antibodies may be IgG1 kappa antibodies. By way of example, the anti-IL-12/IL-23 antibodies of the composition may comprise the heavy chain of SEQ ID NO: 9 and the light of SEQ ID NO: 10. By way of example, the anti-IL-12/IL-23 antibodies of the composition may be ustekinumab.

Any of the pharmaceutical compositions described herein may be prepared into dosage forms in a unit dose suited to fit a dose of the active ingredient. For example, any of the pharmaceutical compositions described herein may be a unit dose comprising no more than 90 mg of the antibodies, such as 90 mg or such as 45 mg. For example, the unit dose may be 45 mg to 90 mg. For some pharmaceutical compositions described herein, the pharmaceutical composition is for a dose of 45 mg of the anti-IL-12/IL-23 antibodies. For some pharmaceutical compositions described herein, the pharmaceutical composition is for a dose of 90 mg of the anti-IL-12/IL-23 antibodies.

Any of the pharmaceutical compositions described herein may be for medical use. For example, a pharmaceutical composition described herein may be accepted by a government regulatory agency. Suitable government regulatory agencies include, without limitation, the FDA, European Medicines Agency (EMA), Pharmaceuticals and Medical Devices Agency (Japan), National Medical Products Administration (China), Health Canada, Medicines and Healthcare Products Regulatory Agency (UK), Central Drug Standard Control Organization (India), and Therapeutic Goods Administration (Australia).

### Methods of Treatment

It is contemplated that the pharmaceutical compositions described herein define attribute ranges that are suitable for medical use. Accordingly, in some embodiments, pharmaceutical compositions as described herein have potency of the anti-IL-12/IL-23 antibodies and safety suitable for medical use, such as for treating inflammatory conditions. For example, the pharmaceutical compositions may be accepted by a government regulatory agency as described herein. For example, the pharmaceutical compositions may be for medical use for a human subject.

Any of the pharmaceutical compositions described herein may be administered to a subject in need of treatment by the anti-IL-12/IL-23 antibodies. Accordingly, contemplated herein are methods comprising administering any of the pharmaceutical compositions to a subject in need of treatment by the anti-IL-12/IL-23 antibodies.

By way of example, the subject may have an inflammatory condition. Examples of inflammatory conditions include psoriasis, psoriatic arthritis, Crohn's disease, and ulcerative colitis. In some embodiments, the inflammatory condition is selected from the group consisting of psoriasis, psoriatic arthritis, ulcerative colitis, and Crohn's disease. Contemplated herein are methods comprising administering any of the pharmaceutical compositions described herein to a subject having an inflammatory condition.

For some methods, the anti-IL-12/IL-23 antibodies are administered at a dose of 45 mg, 90 mg, 130 mg, 260 mg, 390 mg, or 520 mg, including ranges between any two of the listed values, for example, 45 mg-520 mg. As such, any pharmaceutical composition may comprise the anti-IL-12/IL-23 antibodies in a dose of 45 mg, 90 mg, 130 mg, 260 mg, 390 mg, or 520 mg, including ranges between any two of the listed values, for example, 45 mg-520 mg. For some methods, the anti-IL-12/IL-23 antibodies are administered at a dose of 45 mg. For some methods, the anti-IL-12/IL-23 antibodies are administered at a dose of 90 mg.

For any method of treatment comprising a pharmaceutical composition described herein, medical “use” of the pharmaceutical composition is also expressly contemplated, as is the manufacture of a medicament in use for the specified treatment. For example, a method comprising administering a pharmaceutical composition described herein to a subject who has an inflammatory condition, is also contemplated to expressly teach use of the pharmaceutical composition for the treatment of an inflammatory condition in a subject, as well as a manufacture of a medicament for use in the treatment of an inflammatory condition. For any method of treatment described herein (or corresponding use), the subject may be a human subject.

### Methods of Manufacturing

A method of manufacturing pharmaceutical compositions described herein are also contemplated. The method can comprise composition comprising anti-IL-12/IL-23 antibodies as described herein. The method can comprise determining:
(a) an amount that light chains of the composition comprise oxidized W32;
(b) an amount of antibodies of the pharmaceutical composition that comprise isoaspartic acid at position 55 of the heavy chain (EU numbering);
(c) an amount of antibodies of the pharmaceutical composition that are comprised by high molecular weight species selected from dimers and/or oligomers;
(d) an amount of antibodies of the pharmaceutical composition that comprise high mannose; or
(e) an amount of antibodies of the pharmaceutical composition that comprise sialylation; or
(f) an amount of antibodies of the pharmaceutical composition that comprise acidic peak species; or
(optionally) (g) an amount that heavy chains of the composition comprise oxidized W33.
   The methods can further comprise manufacturing the composition for pharmaceutical use when the pharmaceutical composition comprises a molecular attribute (e.g., oxidized light chain W32, isoaspartic acid at position 55 of the heavy chain, high molecular weight species, high mannose, sialylation, acidic peak species, and/or oxidized heavy chain W33) within a specified range of a pharmaceutical composition described herein. As described herein, such pharmaceutical compositions are contemplated to be suitable for medical use.

The methods can further comprise manufacturing the composition for pharmaceutical use when
(a) at least 1% and no more than 2% of the light chains of the composition comprise oxidized W32; and/or
(b) greater than 6% and no more than 14.3% of said antibodies comprise isoaspartic acid at heavy chain position 55, or greater than 6% and no more than 14%, or greater than 7% and no more than 14% of said antibodies comprise isoaspartic acid at heavy chain position 55; and/or
(c) at least 0.4% and no more than 5.0%, or at least 0.4% and no more than 1.0% of said antibodies of the pharmaceutical composition are comprised by high molecular weight species selected from dimers, oligomers, and dimers and oligomers; and/or
(d) at least 1.8% and no more than 6.4%, or greater than 1.8% and no more than 6.4%, or at least 1.8% and no more than 3.7%, or greater than 1.8% and no more than 3.7% of said antibodies of the pharmaceutical composition comprise high mannose; and/or
(e) at least 26.5% and no more than 58%, or at least 26.5% and no more than 29.5%, or at least 26.5% and no more than 29% of said antibodies of the pharmaceutical composition comprise sialylation; and/or
(f) the composition comprises a total of at least 28.0%, or at least 28.0% and no more than 60.3% acidic peak species, or at least 28.0% and no more than 58.3% acidic peak species, or at least 28% and no more than 55.0% acidic peak species as determined by CEX-HPLC; and/or
(g) at least 0.1% and no more than 0.8%, or at least 0.2% and no more than 0.8%, at least 0.1% and no more than 0.4%, or at least 0.2% and no more than 0.4% of the heavy chains of the composition comprise oxidized W33.
   For some methods of manufacturing described herein, the method can further comprise manufacturing the composition for pharmaceutical use when
(a) at least 1% and no more than 2% of the light chains of the composition comprise oxidized W32; and/or
(b) greater than 6.0%, and no more than 14%, or greater than 6.0%, and no more than 12.9% of said antibodies comprise isoaspartic acid at heavy chain position 55; and/or
(c) at least 0.4% and no more than 5.0%, or at least 0.5% and no more than 5.0% of said antibodies of the pharmaceutical composition are comprised by high molecular weight species selected from dimers, oligomers, and dimers and oligomers; and/or
(d) greater than 1.8% and no more than 3.7% of said antibodies of the pharmaceutical composition comprise high mannose; and/or
(e) at least 26.4% and no more than 29.5% of said antibodies of the pharmaceutical composition comprise sialylation; and/or
(f) the composition comprises a total of at least 28.0% and no more than 55.0%, or at least 28.0% and no more than 60.3% acidic peak species as determined by CEX-HPLC.
   The method of manufacturing can comprise determining the amounts of listed attributes according to methods described herein, including, as applicable reduced peptide mapping with MS, SE-UHPLC, HILIC, and/or CEX-HPLC. In some methods, greater than 1% and no more than 2% of the light chains of the composition comprise oxidized W32. In some methods, about 1% of the light chains of the composition comprise oxidized W32. Optionally, the method of manufacturing may further comprise an in vitro potency assay as described herein. Any of the methods of manufacturing may comprise determining two or more of (a), (b), (c), (d), (e), and (f), and manufacturing the composition for pharmaceutical use when two or more of (a), (b), (c), (d), (e), and (f) (as determined) are satisfied. For example, the pharmaceutical composition may be manufactured when (a) and (b); or (a) and (c); or (a) and (d); or (a) and (e); or (a) and (f); or (b) and (c); or (b) and (d); or (b) and (e); or (b) and (f); or (c) and (d); or (c) and (e); or (c) and (f); or (d) and (e); or (d) and (f); or (e) and (f). For example, the pharmaceutical composition may be manufactured when (a) and (b) and (c); or (a) and (b) and (d); or (a) and (b) and (e); or (a) and (b) and (f); or (a) and (c) and (d); or (a) and (c) and (e); or (a) and (c) and (f); or (a) and (d) and (e); or (a) and (d) and (f); or (b) and (c) and (d); or (b) and (d) and (e); or (b) and (e) and (f); or (c) and (d) and (e); or (c) and (d) and (f); or (d) and (e) and (f).

It will be appreciated that any method of manufacturing described herein can comprise determining the amounts of a sample of a composition, and there may not be a need to run an analytical method (such as method described herein) on the entirety of a production lot. The determined amounts can be represented as numerical unit values (e.g., mass or volume units), or as percentages.

### EXAMPLES

### Example 1: Methods Measurement or Determination of Molecular Attributes of Ustekinumab

In the Examples herein, molecular attributes of ustekinumab were measured or determined. “Reference Product” in these Examples refers to STELARA® reference product. In these examples, ustekinumab compositions refer to compositions of ustekinumab according to embodiments herein, which may have different ranges of one or more molecular attribute compared to the Reference Product. The molecular attributes were measured or determined in the Examples according to the following techniques.

Reduced Peptide Mapping with Mass Spectrometry (MS):

Reduced peptide mapping with MS was used to identify molecular attributes of the antibody such as oxidation (e.g., heavy chain W32 oxidation) and aspartic acid isomerization (e.g., isoaspartic acid at position 55 of the heavy chain).

In this method, antibody was reduced with dithiothreitol (DTT) in denaturant and alkylated with iodoacetic acid. Excess reagents were removed by size exclusion-based desalting columns. Digestion with trypsin and/or Asp-N was carried out at 37° C. Subsequently, peptides are separated by RP-HPLC in a Trifluoracetic acid/acetonitrile (TFA/ACN) gradient and monitored by on-line MS and MS/MS data collection. The reduction and alkylation was performed on 500 μg total protein.

Ustekinumab was diluted to a concentration of 1 mg/mL in 7.5M Guanidine HCl (GdnHCl), 0.25M TRIS, 2 mM EDTA, pH 7.5 and denatured at 27±2° C. for 30±5 minutes. The antibody was then reduced with 3 mM dithiothreitol (DTT) at 27±2° C. for 30±5 minutes. The antibody was then S-alkylated with 7 mM iodoacetic acid at 27±2° C. for 15±2 minutes. The antibody was then desalted using a Nap-5 column. The desalted antibody was subsequently digested 0.03 μg/L trypsin at 37±2° C. for 30±5 minutes. The digest was quenched in 0.1 M urea. Peptides produced by the digest were subject to RP-UHPLC on a C18 column over a gradient of 100% mobile phase A+0% mobile phase B, to 5% mobile phase A+95% mobile phase B. The mobile phase gradient was generated with 0% mobile phase B from 0 min to 2 mins, 17% to 23% mobile phase B from 36 mins to 76 mins, 40% mobile phase B from 124 mins to 126 mins, 95% mobile phase B from 126.1 mins to 133 mins; and 0% mobile phase B from 133.1 mins to 144 mins The injection volume was 50 μL. Autosampler was set to 8±2° C., and column compartment was set to 50±2° C. Mobile phase A was 0.05 mM methionine 0.1% TFA in water. Mobile phase B was 0.05 mM methionine 0.1% TFA in acetonitrile. The flow rate was 0.3 mL/min.

Mass spectra were detected using a Thermo Scientific Q Exactive Orbitrap Mass Spectrometer.

### Hydrophilic Interaction Liquid Chromatogram (HILIC):

Oligosaccharides such as glycans of ustekinumab were measured by hydrophilic interaction liquid chromatography (HILIC). Enzymatically released N-linked glycans were labeled with 2-aminobenzoic acid (2-AA) and separated by HILIC (hydrophilic interaction liquid chromatography) with an in-line fluorescence detector. Glycan mixture was injected and the glycans bind to the column in high organic conditions and then eluted as to the glycan hydrophilicity with an increasing gradient of an aqueous ammonium formate buffer. The high-resolution separation was achieved using a 1.7 μm small particle column format and 150 mm column length.

In particular, the antibody was digested at a concentration of no more than 60 mg/mL in PNGase F at 37±2° C. for 120±12 minutes (e.g., 2 μL of PNGase F added to 100 μg of protein in 100 μ/L), releasing glycans from the antibodies. The released glycans were labeled with 2-aminobenzoic acid (2-AA) in a solution comprising 12.5 mg/mL 2-AA and 125 mM sodium cyanoborohydride and 37.5% (v/v) methanol. The glycans were injected onto a 2.1×150 mm, 1.7 μm glycan column over a gradient of 20% mobile phase A 80% mobile phase B, to 80% mobile phase A 20% mobile phase B. The gradient was generated with 20% mobile phase A+80% mobile phase B from 0 to 1 mins, 42% mobile phase A+58% mobile phase B at 110 mins, 80% mobile phase A+20% mobile phase B from 111 mins to 115 mins, and 20% mobile phase A+80% mobile phase B from 120 mins to 125 mins. 2 μl of released and labeled glycan was loaded onto the column. The autosampler temperature was 10° C. The mobile phase A contained 100 mM ammonium formate pH 3.0. The mobile phase B was 100% acetonitrile. The flow rate was 0.4 mL/min. Eluant was monitored by UV at 280 nm.

### Size Exclusion Ultra High-Performance Liquid Chromatography (SE-UHPLC):

Size-exclusion UHPLC separates proteins based on differences in their hydrodynamic volumes. Molecules with larger hydrodynamic volumes elute earlier than molecules with smaller volumes. The samples were loaded onto an SE-UHPLC column, separated isocratically and the eluent is monitored by UV absorbance. Purity was determined by calculating the percentage of each separated component as compared to the total integrated area.

In particular, the UHPLC utilized a 4.6×150 mm column of 1.7 μm particle size. The column was equilibrated with a mobile phase of 250 mM Sodium Chloride 100 mM Sodium Phosphate pH 6.8. 60 μg sample was applied to the column in an injection volume of no more than 20 μL at a sample temperature of 8±5° C. The flow rate was 0.4 mL/min. Eluate was monitored by UV absorbance at 280 nm.

### Cation-Exchange High Performance Liquid Chromatography (CEX-HPLC):

Quantitative purity analysis was performed on the charged variant distribution of ustekinumab by cation-exchange high performance liquid chromatography (CEX-HPLC). Cation Exchange HPLC separates proteins based on differences in their surface charges. At the appropriate pH, positively charged variants of the antibody are separated on a Cation-Exchange column with elution using a salt gradient. The eluent is monitored by UV absorbance. The charged variant distribution is evaluated by determining the peak area of each variant as a percent of the total peak area.

In particular, the antibody was applied to a 4.6 mm×100 mm, 5 μm particle size cation exchange column and eluted along a salt gradient at 30±2° C. and pH 7.0. 60 μg of antibody was loaded onto the cation exchange column in an injection volume of 60 μL. The autosampler temperature set point was 5° C.±3° C. The column temperature set point was 30° C.±2° C. The salt gradient was generated with 5% mobile phase B from 0 min to 2 min, 5% to 12% mobile phase B from 2 min to 21 min, 12% mobile phase B from 21 min to 21.1 min, 12% to 100% mobile phase B from 21.1 min to 21.5 min; 100% mobile phase B from 21.5 min to 23.5 min, 100% to 5% mobile phase B from 23.5 min to 24 min, and to 5% mobile phase B at 24 min to 29 min. Mobile phase A was 20 mM sodium phosphate at pH 7.0. Mobile phase B was 20 mM sodium phosphate, 500 mM sodium chloride at pH 7.0. The mobile phase was applied to the column at a flow rate of 0.8 mL/min. Eluant was monitored by UV absorbance at 280 nm.

### Example 2: Molecular Attributes of Ustekinumab and Reference Product

Molecular attributes were measured in samples of ustekinumab compositions of some embodiments herein. For comparison, molecular attributes of conventional STELARA® reference product (“Reference Product”), which also comprises seminoma, were analyzed. Molecular attributes were determined in ustekinumab composition lots and Reference Product pharmaceutical lots. Molecular attributes were determined according to the methods of Example 1. Percent high molecular weight (HMW) species including oligomers and dimers, was determined by SE-UHPLC. Percent light chain Trp32 (W32) oxidation was determined by reduced peptide mapping with MS. Percent high mannose and percent sialylation were determined by HILIC. Percent CEX acidic peak species were determined by CEX-HPLC. Molecular attribute ranges determined for ustekinumab compositions and Reference Product are shown in Table 1.

TABLE 1
Molecular attribute ranges obtained in ustekinumab
compositions and reference product lots

| | | % IsoAsp | % LC | % High | % | % CEX Acidic |
|---|---|---|---|---|---|---|
| | % HMW | (HC | W320x* | Mannose | Sialylation | Peak |
| Lot | Min/Max | D55) | Min/Max | Min/Max | Min/Max | Min/Max |
| Ustekinumab | 0.5-0.8 | 1-2 | 0-<2 | 2.0-3.7 | 22.3-29.5 | 46.9-55.0 |
| composition | | | | | | |
| Reference | 0.1-0.5 | 0-5 | 0-<1 | 0.7-1.8 | 8.5-26.4 | 14.3-27.6 |
| Product | | | | | | |

*W32 oxidation values were reported as follows: 0% to 0.5% were reported as <1; values of ≥0.5 to 1.5 were reported as 1, and would be understood to refer to values of 1 to <2% when rounding to the nearest digit. It will be understood that the measured values of ustekinumab composition were 0% to <2%.

### Example 3: Molecular Attributes of Ustekinumab Compositions and Reference Product at End-of-Shelf

Molecular attributes were determined in ustekinumab compositions of embodiments herein and Reference Product at end-of-shelf life. For “end-of-shelf life” of the ustekinumab compositions, samples were stored at RSC of 5° C. for 22 months and then moved to ASC of 30° C./75% RH for 2 months in a horizontal orientation.

For “end-of-shelf life” of the Reference Product, samples were stored at RSC of 5° C. for about 36 months and then moved to −70° C. for archiving prior to expiry. The archived samples were tested. Shelf keeping units (SKUs) of the tested ustekinumab compositions of embodiments herein were prefilled syringes (PFS). Shelf keeping units of the tested Reference Product included PFS, 45 mg vial, and 130 mg vial. Molecular attributes with capabilities of changing under storage and stress conditions were measured at end-of-shelf. Minimum and maximum ranges of the listed molecular attributes are shown in Table 2, below.

Molecular attributes were determined in ustekinumab composition lots and Reference Product lots. Molecular attributes were determined according to the methods of Example 1. Percent high molecular weight (HMW) species including oligomers and dimers, was determined by SE-UHPLC. Percent light chain Trp32 (W32) oxidation was determined by reduced peptide mapping with MS. Percent high mannose and percent sialylation were determined by HILIC. Percent CEX acidic peak species were determined by CEX-HPLC.

TABLE 2
Ustekinumab composition and reference product at end-of-shelf

| | % HMW | | % IsoAsp (HC D55) by | | %LC W320x by | |
|---|---|---|---|---|---|---|
| | by SE-UHPLC | | reduced peptide map | | reduced peptide map | |
| Lot | Min | Max | Min | Max | Min | Max |
| Ustekinumab | 0.5 | 0.6 | 5 | 5 | <1 | <1 |
| composition (22 mo) | No. of lots = 5 | | No. of lots = 2 | | No. of lots = 2 | |
| Ustekinumab | 0.6 | 0.8 | 13 | 13 | 1 to <2 | 1 to <2 |
| composition | No. of lots = 5 | | No. of lots = 2 | | No. of lots = 2 | |
| end of shelf | | | | | | |
| (22 mo + 2 mo @30 ° C.) | | | | | | |
| Reference Product | 0.3 | 0.4 | 5 | 6 | <1 | <1 |
| end of shelf | No. of lots = 33 | | No. of lots = 4 | | No. of lots = 4 | |
| (36 mo) | | | | | | |

High molecular wight (HMW) species (including oligomers and dimers), isoaspartic acid at heavy chain position 55, and oxidation of light chain Trp32 (W32) were detected in the ustekinumab composition and in the reference product at their respective end-of-shelf. It was observed that ustekinumab compositions according to some embodiments herein at end-of-shelf contained higher ranges of high molecular weight species, isoaspartic acid at heavy chain position 55, and oxidation of light chain Trp32 (W32) than did the reference product at its respective end-of-shelf. Accordingly, it was contemplated that ustekinumab compositions suitable for medical use in accordance with some embodiments herein can comprise ranges of HMW species, isoaspartic acid at heavy chain position 55, and/or oxidation of light chain W32 that exceed the maximum ranges detected in reference product at end-of-shelf. The combined ranges of molecular attribute in non-expired Reference Product (combined tested lots and end-of-shelf product, across multiple SKUs), along with the combined ranges of molecular attribute observed in ustekinumab compositions of embodiments described herein (combined tested lots and end-of-shelf, across multiple SKUs) are summarized in Table 3, below:

TABLE 3
Combined molecular attribute ranges in ustekinumab
compositions and Reference Product

| | | % IsoAsp | % LC | % High | % | % CEX |
|---|---|---|---|---|---|---|
| | % HMW | (HC D55) | W32Ox | Mannose | Sialylation | Acidic Peak |
| Lot | Min/Max | Min/Max | Min/Max | Min/Max | Min/Max | Min/Max |
| Ustekinumab | 0.5-0.8 | 5-13 | 0 to <2 | 2.0-3.7 | 22.3-29.5 | 46.9-55.0 |
| composition | | | | | | |
| Reference | 0.1-0.5 | 4-6 | <1 | 0.7-1.8 | 8.5-26.4 | 14.3-27.6 |
| product | | | | | | |

### Example 4: Tryptophan Oxidation and Potency

The biological activity of anti-IL-12/IL-23 antibodies such as ustekinumab may change upon exposure to oxidation conditions. Changes in biological activity provide information about the impact of oxidation on the ustekinumab biological function(s) as well as the stability-indicating properties of the in vitro relative activity method. However, the chemical oxidation (AAPH oxidation for tryptophan) condition should not be assumed to necessarily be representative of long-term storage conditions or actual conditions of use. The biological activity of ustekinumab upon exposure to chemical oxidation (AAPH oxidation for tryptophan) was evaluated by IL-12 and IL-23 receptor-ligand binding assays and cell-based reporter gene bioassays. Percent oxidation was determined by reduced peptide map with MS detection as described in Example 1. Potency of AAPH oxidized ustekinumab composition, and non-oxidized controls was determined according to the following assays:

IL-23 Receptor-Ligand Binding Assay: This assay provides a proximal measure of ustekinumab activity and directly reflects the molecular mechanism of action of ustekinumab, which is to bind to the p40 subunit of Interleukin-23 (IL-23) and prevent it from binding to human interleukin-12 receptor 31 (IL-12-R). This method provides a quantitative measure of the ability of ustekinumab to inhibit the binding of IL-23 to IL-12-R. ustekinumab binds to the recombinant IL-23-His ligand (IL-23-His) and inhibits it from binding to biotinylated IL-12 Receptor (IL-12R-biotin). The potency assay is a bead-based Amplified Luminescent Proximity Homogeneous Assay (Alpha) that detects biomolecular interactions. The assay contains two bead types: acceptor beads and donor beads. The donor beads are coated with a hydrogel that contains phthalocyanine, a photosensitizer and streptavidin. The acceptor beads are coated with a hydrogel that contains thioxene derivatives as well as nickel chelate. The donor beads bind to biotinylated IL-12-R through interaction between streptavidin and biotin, and the acceptor beads bind to histidine tagged IL-23 due to the interaction between nickel chelate and histidine. When IL-23-His and biotinylated IL-12-R bind to each other, the acceptor beads and the donor beads are brought into close proximity. When a laser is applied to this complex, ambient oxygen is converted to singlet oxygen by the donor beads. If the beads are in close proximity, an energy transfer to the acceptor beads occurs, resulting in the production of luminescence, which is measured in a plate reader equipped with AlphaScreen® signal detection capabilities. ustekinumab binds to the p40 subunit of IL-23-His and prevents it from binding to biotinylated IL-12-R, thereby decreasing the luminescence output in a dose dependent manner. The test sample activity is determined by comparing the test sample response to the response obtained for the Reference Standard. It will be appreciated that the Receptor-Ligand Binding Assay described in this paragraph is a suitable assay for determining the capability of a composition to inhibit binding of biotinylated IL-12-R immobilized on a donor bead to IL-23-His immobilized on an acceptor bead.

IL-12 Receptor-Ligand Binding Assay: This assay provides a proximal measure of ustekinumab activity and directly reflects the molecular mechanism of action of ustekinumab, which is to bind to the p40 subunit of Interleukin-12 (IL-12) and prevent it from binding to human interleukin-12 receptor 31 (IL-12-R). This method provides a quantitative measure of the ability of ustekinumab to inhibit the binding of IL-12 to IL-12-R. ustekinumab binds to the recombinant IL-12-His ligand (IL-12-His) and inhibits it from binding to biotinylated IL-12 Receptor (IL-12R-biotin). The potency assay is a bead-based Amplified Luminescent Proximity Homogeneous Assay (Alpha) that detects biomolecular interactions. The assay contains two bead types: acceptor beads and donor beads. The donor beads are coated with a hydrogel that contains phthalocyanine, a photosensitizer and streptavidin. The acceptor beads are coated with a hydrogel that contains thioxene derivatives as well as nickel chelate. The donor beads bind to biotinylated IL-12-R through interaction between streptavidin and biotin, and the acceptor beads bind to histidine tagged IL-12 due to the interaction between nickel chelate and histidine. When IL-12-His and biotinylated IL-12-R bind to each other, the acceptor beads and the donor beads are brought into close proximity. When a laser is applied to this complex, ambient oxygen is converted to singlet oxygen by the donor beads. If the beads are in close proximity, an energy transfer to the acceptor beads occurs, resulting in the production of luminescence, which is measured in a plate reader equipped with AlphaScreen® signal detection capabilities. ustekinumab binds to the p40 subunit of IL-12-His and prevents it from binding to biotinylated IL-12-R, thereby decreasing the luminescence output in a dose dependent manner. The test sample activity is determined by comparing the test sample response to the response obtained for the Reference Standard. It will be appreciated that the Receptor-Ligand Binding Assay described in this paragraph is a suitable assay for determining the capability of a composition to inhibit binding of biotinylated IL-12-R immobilized on a donor bead to IL-23-His immobilized on an acceptor bead.

Inhibition of IL-23-mediated Signaling Assay: IL-23 induces the signal transducer and activation of transcription (STAT) signaling pathway upon binding to the heterodimeric receptor composed of IL-23R and IL-12Rβ1 chains. ustekinumab binds to the p40 subunit of IL-23 and prevents it from binding IL-12Rβ1, therefore preventing the activation of the STAT signaling pathway. In this assay, cells expressing IL-23R containing IL-12Rβ1, IL-23R, and a Sis-inducible element (SIE)-response element (RE) luciferase gene (Luc2P) are utilized to measure the IL-23 induced STAT signaling pathway. In the presence of IL-23, the cells are induced to generate a bioluminescent signal that can be detected with addition of luminescence reagent. The addition of ustekinumab inhibits IL-23 mediated signaling pathway resulting in a reduction in the amount of luminescence detected. The test sample activity is determined by comparing the test sample response to the response obtained for the Reference Standard.

Inhibition of IL-12-mediated Signaling Assay: Binding of IL-12 to its heterodimeric receptor composed of IL-12Rβ2 and IL-12Rβ1 chains induces the signal transducer and activation of transcription (STAT) signaling pathway. Ustekinumab binds to the p40 subunit of IL-12 and prevents it from binding IL-12Rβ1, therefore preventing the activation of the STAT signaling pathway. In this assay, cells expressing IL-12Rβ1, IL-12Rβ2, and a Sis-inducible element (SIE)-response element (RE) luciferase gene (Luc2P) are utilized to measure the IL-12 induced STAT signaling pathway. In the presence of IL-12, the cells are induced to generate a bioluminescent signal that can be detected with addition of luminescence reagent. The addition of ustekinumab inhibits IL-12 mediated signaling pathway resulting in a reduction in the amount of luminescence detected. The test sample activity is determined by comparing the test sample response to the response obtained for the Reference Standard.

Levels of oxidized LC W32 at hour 24 after AAPH oxidation for tryptophan, as well as the relative potency of AAPH-oxidized ustekinumab and non-oxidized controls as determined by the potency assays described herein is shown in Table 4.

TABLE 4
Relative Activity by Chemical Oxidation (AAPH Oxidation for Tryptophan, hour 24)

| | | % IL-23 | % Inhibition of | % IL-12 | % Inhibition of | |
|---|---|---|---|---|---|---|
| Time | Oxidized LC | Receptor-ligand | IL-23-mediated | Receptor-ligand | IL-12-mediated | |
| Point | W32 at 24 h | Binding | Signaling | Binding | Signaling | |
| (Day) | AAPH (%) | (% CV) | (% CV) | (% CV) | (% CV) | |
| Negative | 1 | 104 (7) | 95 (2) | 100 (6) | 103 | (3) |
| control | | | | | | |
| Hour 24 | 44 | 101 (6) | 54 (7) | 106 (7) | 66 | (2) |

These data show that tryptophan oxidation, resulting in oxidation of 44% of LC W32, impacted potency of ustekinumab, as measured by the IL-23-mediated signaling and IL-12-mediated signaling.

### Example 5: High Mannose Modeling and Clearance

A modeling approach was used to estimate the potential impact of an increase in % HM on the clearance of ustekinumab. The model assumed a half-life of 24.5 days (based on half-life (t½) of the antibody in a clinical study (e.g., single dose 90 mg SC in healthy adults) and an increased rate constant of 0.035 Day⁻¹ for the HM form of the antibody based on the HM % decrease rate of a reference IgG2 antibody following single IV dose. The increased rate constant of the reference IgG2 antibody HM form has the highest value analyzed to date and was chosen as a conservative estimate of the ustekinumab HM half-life. The PK profile was modeled up to 122.5 days (the equivalent of 5 half-lives) and no correction for preferential pairing was used. High mannose levels detected in ustekinumab pharmaceutical compositions of some embodiments herein, as well as in STELARA® product (Reference Product) are shown in Table 5, along with the estimated increase in clearance per the model described above.

TABLE 5

| Ustekinumab HM levels (%) | Reference Product | Estimated increase |
|---|---|---|
| for compositions herein | HM level (%) | in the clearance (%) |
| 1.8 | 0.7 | 0.6 |
| 3.7 | 0.7 | 1.6 |
| 19.3 | 0.7 | 10 |

Based on this analysis, it was contemplated that ustekinumab pharmaceutical compositions can comprise up to 19.3% high mannose with no more than a 10% increase in clearance compared to reference antibody containing 0.7% high mannose. Additionally, pharmaceutical compositions comprising ustekinumab of embodiments herein can comprise up to 3.7% high mannose with no more than a 1.6% increase in clearance, compared to Reference Product ustekinumab containing 0.7% high mannose.

### Example 6.1: Clinical Impact of Sialylation

The impact of elevated sialylation levels on subjects treated with ustekinumab was assessed. Sialylation of ustekinumab and the US and EU lots of Reference Product material was determined using HILIC as described herein. From these data, subjects were exposed to different levels of sialylation during clinical administration in the assessed phase 1 clinical trials. The sialylated species exposure was calculated for lots suitable for medical use at the time of clinical administration (See, e.g., International Patent App. Pub. No. WO2022132982).

TABLE 6
Reference Product and ustekinumab sialylation levels

| | Sialylation | Sialylated species |
|---|---|---|
| Material | (%) | Exposure (mg) |
| Reference Product (US) PFS | 23.3 | 20.9 |
| Reference Product (EU) PFS | 23.0 | 20.7 |
| Ustekinumab Composition PFS | 29.0 | 26.1 |
| Ustekinumab Composition PFS/ | 28.9 | 26.0 |
| Autoinjector | | |

It was calculated that reference pharmaceutical compositions of Reference Product have 23.3% sialylation (US) and 23.0% sialylation (EU) which correspond to 20.9 mg and 20.7 mg of sialylated drug product, respectively, in a 90 mg dose formulation. The ustekinumab composition of embodiments herein has 29.0% sialylation and 28.9% (Autoinjector) which corresponds to 26.1 mg and 26.0 mg of sialylated drug product, respectively, in a 90 mg dose formulation. Adverse events (AE) analyzed include occurrence of Binding ADA (anti-drug antibody), Headache, Diarrhea, Nausea, Back pain, COVID-19, Pruritus, Myalgia, Dizziness, Oropharyngeal pain, Abdominal pain, Nasopharyngitis, and Fatigue. None of the adverse events were observed to correlate with clinical exposure to sialylated drug product. Accordingly, it is contemplated that the sialylation levels assessed (up to 29.0%) herein can be tolerated in ustekinumab compositions that remain clinically suitable.

### Example 6.2: Additional Analysis of Clinical Impact of Sialylation

In addition to the analysis described in Example 6.1, phase 3 clinical trial data were assessed. The sialylated species exposure was calculated for lots suitable for medical use at the time of clinical administration (See, e.g., International Patent App. Pub. No. WO2022132982). It was calculated that reference pharmaceutical compositions of Reference Product had 24.45% sialylation (EU) which correspond to 22.00 mg of sialylated drug product in a 90 mg dose formulation. The ustekinumab composition of embodiments herein had 29.00% sialylation, which corresponds to 26.10 mg of sialylated drug product in a 90 mg dose formulation. Patients were dosed in this phase 3 study at 45 mg and 90 mg based on their weight. A maximum exposure of 26.10 mg sialylated ustekinumab species corresponds to 29.00% sialylated ustekinumab species in a 90 mg dose and 58.00% in a 45 mg dose. For clarity, 29.00% sialylated ustekinumab of embodiments herein was present in the 90 mg dose that was administered to patients and 58.00% sialylated ustekinumab was calculated based on the relative gravimetric amount of attribute exposure in the 45 mg dose of ustekinumab. Adverse Events (AE) analyzed included occurrence of ADA (anti-drug antibody), COVID-19, nasopharyngitis, upper respiratory tract infection, hypertension, headache, proteinuria, urinary tract infection, blood pressure increase, arthralgia, skin laceration, and back pain. None of the adverse events were observed to correlate with clinical exposure to sialylated drug product. Binding anti-drug antibodies were detected for 175 patients and no unusual associations were observed between binding anti-drug antibodies and sialylated drug product exposure. Additionally, separate analyses for patients who were administered ustekinumab only vs. patients who were administered the reference product only showed that sialylated drug products exposure was not associated with binding antidrug antibodies. Accordingly, it is contemplated that the sialylation levels assessed herein (up to 58.00%) can be tolerated in ustekinumab compositions that remain clinically suitable.

### Example 7: Molecular Attributes of Additional Lots of Ustekinumab Compositions at End-of-Shelf

Molecular attribute data were collected for additional lots of ustekinumab compositions as described herein at end-of-shelf. Data were collected for both an 24 month end-of-shelf (22 months at 2-8° C. plus 2 months at 30° C.), and a 36 month end-of-shelf (34 months at 2-8° C. plus 2 months at 30° C.). Molecular attributes were measured by reduced peptide map (IsoAsp HC D55 and LC W32Ox), SE-UHPLC (% HMW), and CEX-HPLC (% Acidic Peak) using the methods described in Example 1.

Molecular attributes were determined according to the methods of Example 1. Percent high molecular weight (HMW) species including oligomers and dimers, was determined by SE-UHPLC. Percent heavy chain position 55 (D55) isoaspartic acid, and percent light chain Trp32 (W32) oxidation were determined by reduced peptide mapping with MS. Percent CEX acidic peak species were determined by CEX-HPLC. Molecular attribute ranges determined for ustekinumab compositions and Reference Product are shown in Table 7.

TABLE 7
Additional lots of ustekinumab composition at end-of-shelf

| | % HMW by SE- | | % IsoAsp (HC D55) by | | % LC W32Ox by | | % Acidic Peaks by | |
|---|---|---|---|---|---|---|---|---|
| End-of-shelf | UHPLC | | reduced peptide map | | reduced peptide map | | CEX-HPLC | |
| Conditions | Min | Max | Min | Max | Min | Max | Min | Max |
| 22 mo at 2-8° C. | 0.1 | 0.9 | 13% | 14% | 1% | 2% | 57.5 | 60.3 |
| plus 2 mo at 30° C. | No of lots = 10 | | No of lots = 4 | | No of lots = 4 | | No of lots = 10 | |
| 34 mo at 2-8° C. | 0.8 | 0.8 | 14% | 14% | 1% | 1% | 59.2 | 59.2 |

| | | | | |
|---|---|---|---|---|
| plus 2 mo at 30° C. | No of lots = 1 | No of lots = 2 | No of lots = 2 | No of lots = 1 |

Accordingly, it was observed that the ustekinumab compositions as described herein can comprise up to at least 0.9% HMW species (as determined by SE-UHPLC), 14% IsoAsp (HC D55), 2% LC W32Ox, and/or 60.3% acidic peaks, and be suitable for medical use.

### Example 8: Desialylation of Ustekinumab Composition

To assess impacts or absence of impacts of sialic acid in ustekinumab compositions as described herein, stability and immunogenicity of ustekinumab comprising sialylation was compared with immunogenicity and stability of ustekinumab that had been disialylated.

With the desialylation method described herein, the % sialylation level of ustekinumab in accordance with some embodiments herein can be reduced from 28% to 2%. For Reference Product, this level can be decreased from 20% to 6%. As described herein, readouts for both a cell line assay and a PBMC assay confirmed that there is no increased risk of immunogenicity for ustekinumab compositions comprising up to 28% sialylation, as no difference was detected in response to ustekinumab with and without sialylation. As further described herein, a stability study of force degraded ustekinumab further that the sialylation level of up to 28% has no negative impact on protein stability under both light and thermal stress condition.

Desialylation with Sialidase

Desialylation of Reference Product and ustekinumab composition (as described herein) was achieved by enzymatic digestion under native condition. After removal of terminal sialic acid, desialylated protein was purified with Protein A enrichment, then reformulated and concentrated to formulation buffer using molecular weight cut-off filters.

In particular, to completely remove sialic acid, the ustekinumab composition was digested at a concentration of no more than 5 mg/mL with sialidase (e.g., 180 μL of sialidase added to 2.5 mg of protein in 5 mg/mL, preferred to use a 51 kDa version of sialidase to avoid steric hindrance) at 37±2° C. for at least 1 day (e.g. 1 day incubation for ustekinumab composition and 7 day incubation for Reference Product). The digestion mixture was loaded onto pre-conditioned Protein A column for desialylated protein purification follow vendor recommended protocol. For example, 2.5 mL of 5 mg/mL desialylated protein solution was diluted with 2.5 mL of Protein A binding buffer (25 mM Tris, 100 mM NaCl, pH 7.4) and loaded onto 1 mL Nab Protein A Plus column. Wash column with 15 mL of binding buffer, then captured protein from column with 5 mL of elution buffer (100 mM Sodium Acetate, pH 3.6). Purified IgG was immediately buffer exchanged with 6.4 mM L-histidine, 7.6% sucrose, pH 6 by 10 kDa MWCO filter and concentrated with same filter. The estimated concentration after buffer exchange should be 90-100 mg/mL. Finally, 0.1% PS80 was spiked in protein solution to make the final PS80 concentration at 0.002-0.006%.

### Force Degradation of Desialylated Protein

Force degradation studies were executed to assess the stability of desialylated protein.

For thermal stress study, the desialylated ustekinumab composition and Reference Product were aliquoted in vials (e.g. 200 uL per time point) and degraded up to 14 days in a 50° C. incubator. The day 0 vial remained at 2-8° C. until analysis. The aliquoting vial was removed at each time point and remained at 2-8° C. until analysis. The time points for thermal degradation were day 0, 3, 7, 10 and 14.

For photo stress study, the desialylated ustekinumab and Reference Product were aliquoted in vials (e.g. 200 uL per time point) and degraded using 8.0 klux hours of cool white (visible) light for a total of 1.2 million lux (Mlux) hours. All vials, including the foil wrapped controls, were added to the photo stability chamber set to 25° C. and 8 klux. The day 0 vial remained at 2-8° C. in dark until analysis. The aliquoting vial was removed at each time point and remained at 2-8° C. until analysis, and the foil wrapped controls were stored in the chamber until the last time point. The time points for thermal degradation were day 0, 1, 3 and 6.3 (1.2 million lux hours).

Desialylated samples were compared to controls comprising 28% sialylation (ustekinumab composition) or 20% sialylation (reference product). To investigate the stability of force degraded materials, all samples were submitted to SE-UHPLC analysis follow protocol described in Example 1 (SE-UHPLC section), as well as reduced peptide mapping analysis showed in Example 1 (reduced peptide mapping with mass spectrometry section).

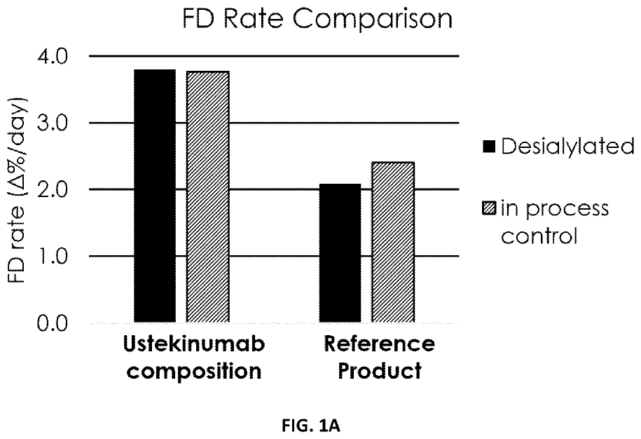

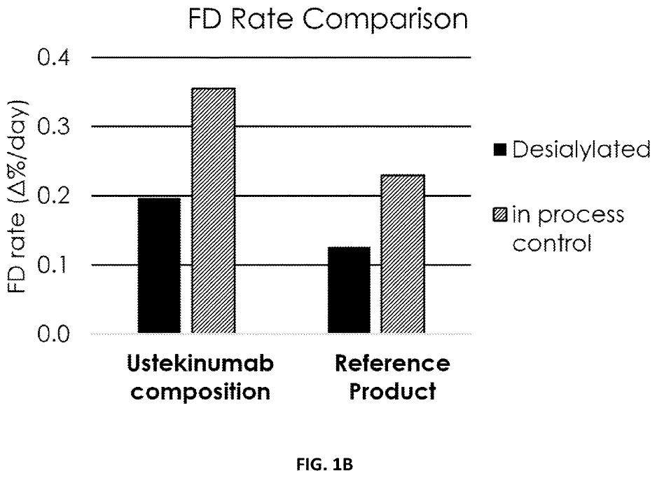

Results for light stress degradation are shown in FIG. 1A. Results for thermal degradation are shown in FIG. 1B. From these experiments, it was concluded that sialylation levels of up to 28% have no negative impact on protein stability (based on SEC analysis) in ustekinumab compositions.

### PBMC Immunogenicity Assay

To investigate the immunogenicity of ustekinumab sialic acid samples in the in vitro human cell-based assay known as the PBMC Assay was performed. The PBMC assay comprises PBMCs from five separate individual donors being treated with the various conditions which are depleted for CD8 T cells and comprise of PBMCs depleted of CD8 T cells. Evaluations are in CD4 T cells. After treatment, this assay allows for the following endpoint readouts:
20-hour Luminex multiplex cytokine profiling to assess early immune activation as measured by Luminex cytokine profiling (11-plex)
7-day CD4 T-cell activation as determined by expression CD25 and HLA-DR T cell activation markers on the surface of CD4 T cells by flow cytometry
7-day CD4 T-cell proliferation as determined by dilution of Cell Trace Violet proliferation dye measure by flow cytometry
7-day Luminex multiplex cytokine profiling to assess early immune activation as measured by Luminex cytokine profiling (12-plex)

Each of these assays at the corresponding endpoints, along with corresponding positive and negative controls, can be used to assess the immunogenicity risk of attributes by 1) assessing the impact of attributes on T cell proliferation, 2) assessing the impact of attributes on T cell activation, 3) assessing the impact of attributes on cytokine release at 20 hours and 7 days post stimulation with the attributes, as well as other treatments and necessary controls. All PBMC donors were treated with a variety of samples. For the analysis of data ustekinumab TS10 sialylated was compared to ustekinumab TS10 desialylated; TLR 1-9 Agonist mix, TLR 1-9 Agonist mix and ustekinumab TS10 sialylated, and TLR 1-9 Agonist mix and ustekinumab TS10 desialylated were compared to each other; free sialic acid, free sialic acid and ustekinumab TS10 sialylated, and free sialic acid and ustekinumab TS10 desialylated were compared to each other. The main goal was to compare sialylated and desialylated ustekinumab promoted immunogenic stimulation as determined by assessment of 1) T cell proliferation, 2) T cell activation, 3) cytokine release at 20 hours and 7 days post stimulation. As a control, samples of antibody 10E4, an antibody with an established propensity to form aggregates, was also run in the assay under both aggregated and non-aggregated conditions. Formulation buffer (FB) and acetate pH 5 (A5) buffer were also run as controls.

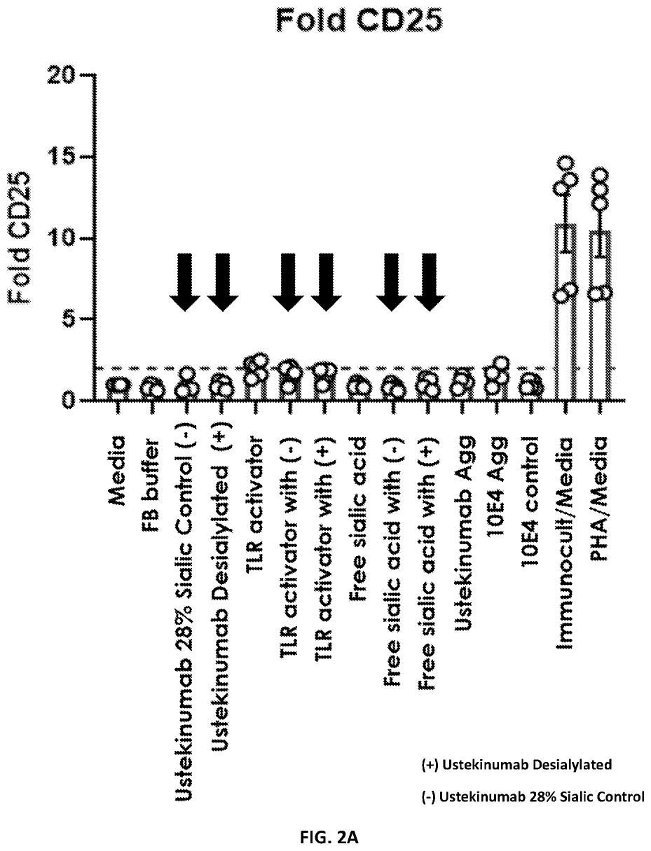

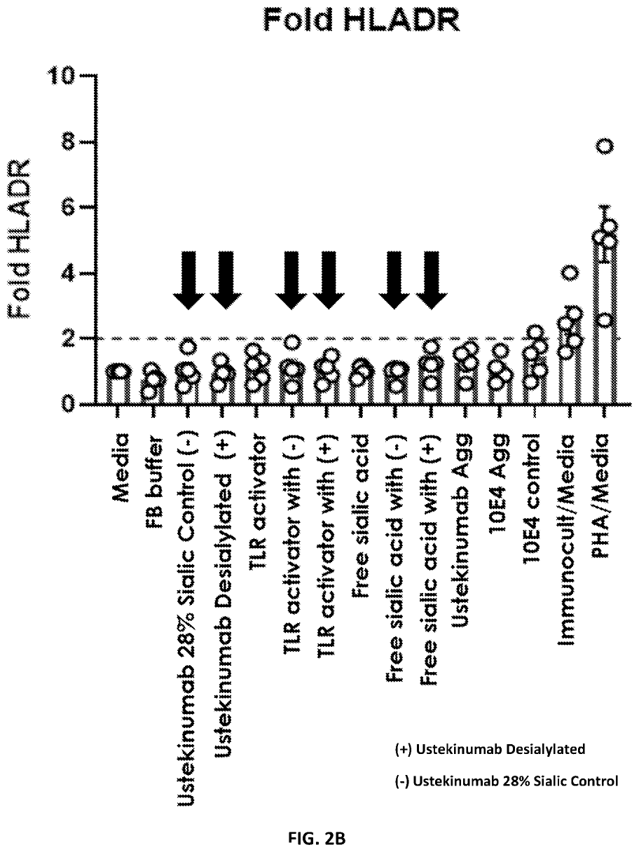

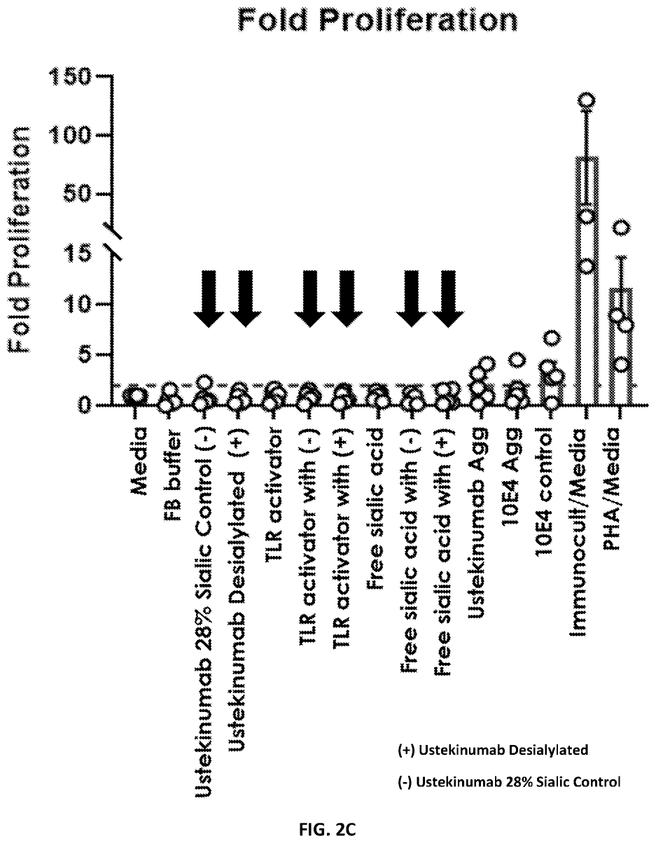

Compared to ustekinumab composition comprising 28% sialylation, desialylation of the ustekinumab composition did not impact T cell activation as measured by CD25 (FIG. 2A) and HLA-DR expression (FIG. 2B) or proliferation (FIG. 2C), indicating that the ustekinumab compositions as described herein may comprise sialylation of up to at least 28% without an impact on T cell activation.

### Cell Line Immunogenicity Assay

To investigate any immunogenicity of the ustekinumab sialic acid attribute, ustekinumab composition comprising 28% sialic acid (“ustekinumab 28% sialic”), was treated with neuramidase to create a desialylated sample (“ustekinumab desialylated”). These samples in conjunction with comparative controls are applied for immunogenicity risk assessment using a human Cell Line Assay model system. The cell line assay consists of three cell lines with an NFkB/AP-1 transcriptional controlled embryonic alkaline phosphatase (SEAP) reporter: THP-1 Blue (monocytes), Ramos-Blue (B-cells) and Raw-Blue (macrophages). Each of these cell lines has different types of Toll-like-receptors (TLRs). TLRs are known to induce NFkB/AP-1 transcriptional activation due to antigen/ligand induced activation of TLRs. If TLRs are engaged, SEAP will be produced and secreted by the cell lines. The SEAP then interacts with QUANTI-BLUE to produces a colorimetric change that can be measured by absorbance- and is a measure of NFkB activity and thus inflammation. For the analysis of data ustekinumab 28% sialic was compared to ustekinumab desialylated; TLR 1-9 Agonist mix, TLR 1-9 Agonist mix and ustekinumab 28% sialic, and TLR 1-9 Agonist mix and ustekinumab desialylated were compared to each other; free sialic acid, free sialic acid and ustekinumab 28% sialic, and free sialic acid and ustekinumab desialylated were compared to each other. The experiment compared ustekinumab 28% sialic and desialylated ustekinumab-promoted immunogenic stimulation as determined by assessment of SEAP release under the NFkB/AP-1 transcriptional control. As a control, samples of antibody 10E4, an antibody with an established propensity to form aggregates which can result in immunogenicity, was also run in the assay under both aggregated and non-aggregated conditions.

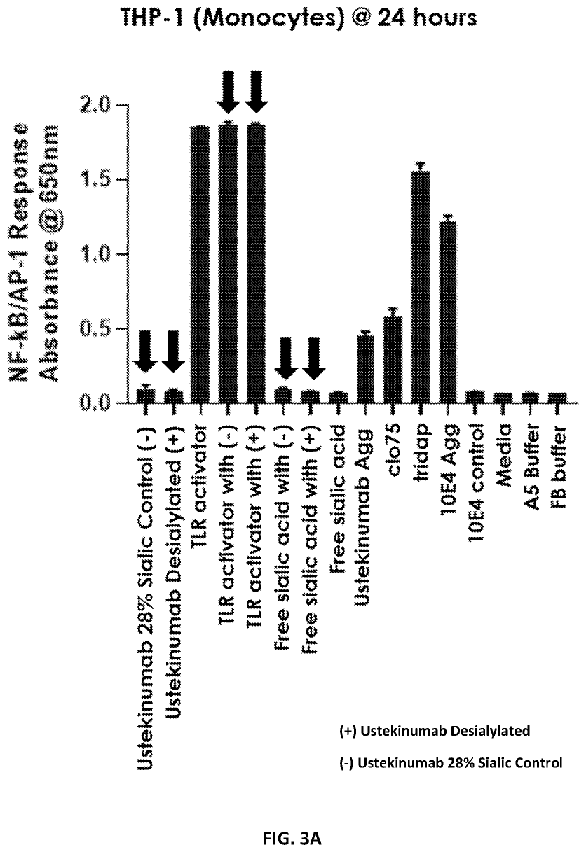

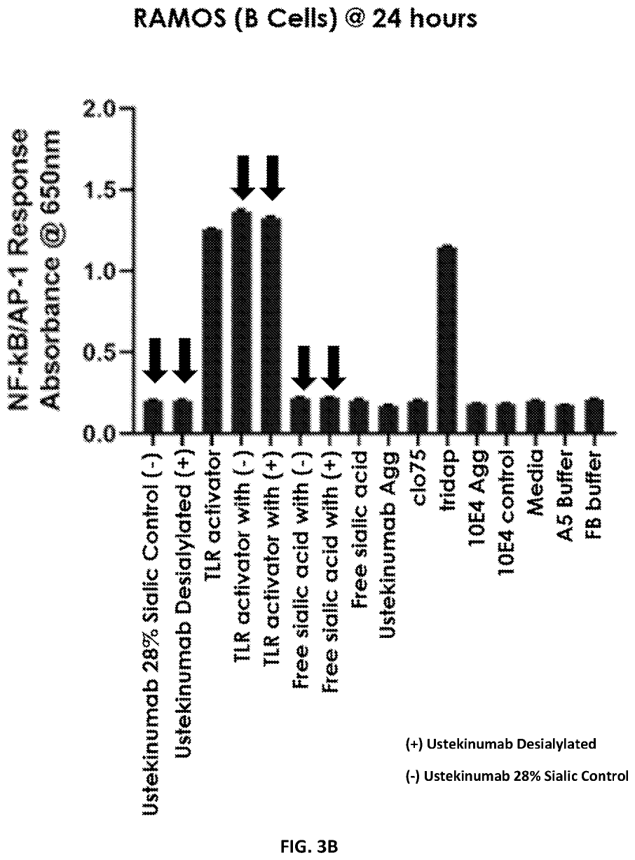

Desialylation of the ustekinumab as described herein did not impact induce NFkB activation in THP-1 cell lines (FIG. 3A) and Ramos cell lines (FIG. 3B), indicating that the ustekinumab as described herein may comprise sialylation of up to at least 28% without an impact on T cell activation.

### Example 9: Analysis of High Molecular Weight Species

High molecular weight (HMW) species of ustekinumab in clinical pharmaceutical compositions after different storage durations at 5° C. were determined by SE-UPLC as described in Example 1. From these data, stability profiles modeling the rate of HMW species formation over time at 5° C. were developed. Based on percentage change per month as shown in Table 8, the HMW species exposure was calculated for lots suitable for medical use at the time of clinical administration (See International Patent App. No. PCT/US21/63641). Data were calculated for lots used in a Phase 3 clinical trial.

TABLE 8
Reference Product and ustekinumab
HMW Common Slope Estimate %

| | Common slope Estimate |
|---|---|
| Material | (units/month) HMW % |
| Reference Product (EU) | 0.001 |
| Ustekinumab composition PFS | 0.008211 |

It was calculated that pharmaceutical compositions comprising ustekinumab in accordance with embodiments herein for clinical administration comprised up to 0.47 mg HMW species at the time of clinical administration. It was calculated that pharmaceutical compositions comprising Reference Product for clinical administration comprised up to 0.28 mg HMW species (0.32% HMW species) at the time of clinical administration. HMW species of 0.47 mg for ustekinumab in accordance with embodiments herein, and HMW species of 0.28 mg for Reference Product corresponds to 0.52% and 0.32% HMW species, respectively, in a 90 mg dose formulation. Patients were dosed in the Phase 3 study at 45 mg and 90 mg based on their weight. A maximum exposure of 0.47 mg corresponds to 0.52% in a 90 mg dose and 1.04% in a 45 mg dose. For clarity, 0.52% HMW species was present in the 90 mg dose of ustekinumab composition that was administered to patients and the 1.04% HMW specie was calculated based on the relative gravimetric amount of attribute exposure in the 45 mg dose. As such, it is contemplated herein that pharmaceutical compositions comprising ustekinumab in accordance with embodiments herein can comprise up to 1.04% HMW species. Adverse Events (AE) analyzed included occurrence of ADA (anti-drug antibody), COVID-19, nasopharyngitis, upper respiratory tract infection, hypertension, headache, proteinuria, urinary tract infection, blood pressure increase, arthralgia, skin laceration, and back pain. None of the adverse events were observed to correlate with clinical exposure to HMW species in the phase 3 clinical study. Binding anti-drug antibodies were detected for 175 patients and no unusual associations were observed between binding anti-drug antibodies and HMW species exposure. Additionally, separate analyses for patients who were administered ustekinumab only vs. patients who were administered the reference product only showed that HMW species exposure of both drug products was not associated with binding antidrug antibodies. Accordingly, it is contemplated that the HMW species level assessed herein can be tolerated in ustekinumab compositions that remain clinically suitable.

### Example 10: Analysis of Aspartic Acid Isomerization (IsoAsp) Species

Heavy chain aspartic acid (D55) isomerization species of ustekinumab in accordance with embodiments herein and Reference Product in clinical pharmaceutical compositions after different storage durations at 5° C. were determined by reduced peptide mapping as described in Example 1. Based on percentage change per month as in Table 9, the isomerization species at heavy chain position 55 (D55) exposure was calculated for lots suitable for medical use at the time of clinical administration (See International Patent App. No. PCT/US21/63641). Data were calculated for lots used in a Phase 3 clinical trial.

TABLE 9
Reference Product and ustekinumab D55 isomerization
species Common Slope Estimate %

| | Common slope Estimate |
|---|---|
| Material | (units/month) HMW % |
| Reference Product (EU) | 0.0 |
| Ustekinumab composition PFS | 0.00246 |

It was calculated that pharmaceutical compositions comprising ustekinumab according to embodiments herein for clinical administration comprised up to 6.44 mg D55 isomerization at the time of clinical administration. It was calculated that pharmaceutical compositions comprising Reference Product for clinical administration comprised up to 2.70 mg heavy chain D55 isomerization (3.00% heavy chain D55 isomerization) at the time of clinical administration. For clarity, 6.44 mg in ustekinumab compositions and 2.70 mg in Reference Product of heavy chain D55 isomerization corresponds to 7.15% and 3.00%, respectively, in a 90 mg dose formulation. Patients were dosed in the Phase 3 study at 45 mg and 90 mg based on their weight. A maximum heavy chain D55 isomerization species exposure of 6.44 mg corresponds to 7.15% in a 90 mg dose and 14.30% in a 45 mg dose. For clarity, 7.15% heavy chain D55 isomerization was present in the 90 mg dose of ustekinumab (in accordance with compositions of embodiments herein) that was administered to patients and 14.30% heavy chain D55 isomerization was calculated based on the relative gravimetric amount of attribute exposure in the 45 mg dose. Accordingly, it is contemplated herein that pharmaceutical compositions comprising ustekinumab in accordance with embodiments herein can comprise up to 14.30% heavy chain D55 isomerization. Adverse Events (AE) analyzed included occurrence of ADA (anti-drug antibody), COVID-19, nasopharyngitis, upper respiratory tract infection, hypertension, headache, proteinuria, urinary tract infection, blood pressure increase, arthralgia, skin laceration, and back pain. None of the adverse events were observed to correlate with clinical exposure to the heavy chain D55 isomerization species in the Phase 3 clinical study. Binding anti-drug antibodies were detected for 175 patients and no unusual associations were observed between binding anti-drug antibodies and tryptophan oxidation exposure. Accordingly, it is contemplated that the heavy chain D55 isomerization species assessed herein can be tolerated in ustekinumab compositions that remain clinically suitable.

### Example 11: Analysis of High Mannose Species

High Mannose (HM) species of ustekinumab in accordance with embodiments herein and Reference Product in clinical pharmaceutical compositions after different storage durations at 5° C. were determined by HILIC N-glycan mapping procedure as described in Example 1. HM species exposure was calculated for lots suitable for medical use at the time of clinical administration (see International Patent App. No. PCT/US21/63641). Data were calculated for lots used in a Phase 3 clinical trial. It was calculated that pharmaceutical compositions comprising ustekinumab in accordance with embodiments herein for clinical administration comprised up to 2.87 mg HM species at the time of clinical administration. It was calculated that pharmaceutical compositions comprising Reference Product for clinical administration comprised up to 0.93 mg (1.03%) HM species at the time of clinical administration. For clarity, 2.87 mg in ustekinumab and 0.93 mg in Reference Product of HM species corresponds to 3.18% and 1.03%, respectively, in a 90 mg dose formulation. Patients were dosed in the Phase 3 study at 45 mg and 90 mg based on their weight. A maximum exposure of 2.87 mg HM corresponds to 3.18% in a 90 mg dose and 6.36% in a 45 mg dose of ustekinumab. For clarity, 3.18% HM species was present in the 90 mg dose of ustekinumab composition that was administered to patients and the 6.36% HM species was calculated based on the relative gravimetric amount of attribute exposure in the 45 mg dose. Accordingly, it is contemplated herein that pharmaceutical compositions comprising ustekinumab in accordance with embodiments herein can comprise up to 6.36% HM species. Adverse Events (AE) analyzed included occurrence of ADA (anti-drug antibody), COVID-19, nasopharyngitis, upper respiratory tract infection, hypertension, headache, proteinuria, urinary tract infection, blood pressure increase, arthralgia, skin laceration, and back pain. None of the adverse events were observed to correlate with clinical exposure to the HM species in the Phase 3 clinical study. Binding anti-drug antibodies were detected for 175 patients and no unusual associations were observed between binding anti-drug antibodies and HM species exposure. Accordingly, it was contemplated that HM species can be tolerated in ustekinumab pharmaceutical compositions that remain clinically suitable. Accordingly, it is contemplated that the HM species assessed herein (up to 6.36%) can be tolerated in ustekinumab compositions that remain clinically suitable.

### Example 12: Analysis of Tryptophan Oxidation (TrpOx) Species

Tryptophan oxidation of ustekinumab in accordance with embodiments herein and Reference Product in clinical pharmaceutical compositions after different storage durations at 5° C. were determined by reduced peptide mapping as described in Example 1. The tryptophan oxidized species exposure was calculated for lots suitable for medical use at the time of clinical administration (see International Patent App. No. PCT/US21/63641). Data were calculated for lots used in a Phase 3 clinical trial. It was calculated that pharmaceutical compositions comprising ustekinumab according to embodiments herein for clinical administration comprised up to 0.90 mg oxidized LC W32, and 0.36 mg oxidized HC W33, respectively at the time of clinical administration. It was calculated that pharmaceutical compositions comprising Reference Product for clinical administration comprised up to 0.27 mg oxidized LC W32, and 0.05 mg oxidized HC W33, at the time of clinical administration. 0.90 mg in ustekinumab and 0.27 mg in Reference Product of oxidized LC W32 species corresponds to 1.00%, and 0.30%, respectively, in a 90 mg dose formulation. 0.36 mg in ustekinumab and 0.05 mg in Reference Product of oxidized HC W33 corresponds to 0.40%, and 0.05%, respectively, in a 90 mg dose formulation. Patients were dosed in the Phase 3 study at 45 mg and 90 mg based on their weight. For oxidized LC W32, a maximum exposure of 0.90 mg corresponds to 1.00% in a 90 mg dose and 2.00% in a 45 mg dose; for oxidized HC W33, a maximum exposure of 0.36 mg corresponds to 0.40% in a 90 mg dose and 0.80% in a 45 mg dose. For clarity regarding oxidized LC W32, 1.00% was present in the 90 mg dose that was administered to patients and the 2.00% was calculated based on the relative gravimetric amount of attribute exposure in the 45 mg dose. For clarity regarding HC W33, 0.40% was present in the 90 mg dose that was administered to patients and the 0.80% was calculated based on the relative gravimetric amount of attribute exposure in the 45 mg dose. Accordingly, it is contemplated herein that pharmaceutical compositions comprising ustekinumab in accordance with embodiments herein can comprise up to 2% oxidized LC W32. Accordingly, it is contemplated herein that pharmaceutical compositions comprising ustekinumab in accordance with embodiments herein can comprise up to 0.8% oxidized HC W33. Adverse Events (AE) analyzed included occurrence of ADA (anti-drug antibody), COVID-19, nasopharyngitis, upper respiratory tract infection, hypertension, headache, proteinuria, urinary tract infection, blood pressure increase, arthralgia, skin laceration, and back pain. None of the adverse events were observed to correlate with clinical exposure to tryptophan oxidized species in the Phase 3 clinical study. Binding anti-drug antibodies were detected for 175 patients and no unusual associations were observed between binding anti-drug antibodies and tryptophan oxidation exposure. Accordingly, it was contemplated that LC W32 and HC W33 oxidized species can be tolerated in ustekinumab pharmaceutical compositions that remain clinically suitable. Accordingly, it is contemplated that the LC W32 and HC W33 oxidized species assessed herein can be tolerated in ustekinumab compositions that remain clinically suitable.

### Example 13: Analysis of CEX-HPLC Acidic Peak Species

CEX acidic peak species levels in ustekinumab compositions according to embodiments herein and Reference Product in clinical pharmaceutical compositions were determined by CEX-HPLC as described in Example 1. The CEX acidic peaks (or “acidic peak species”) exposure was calculated for lots suitable for medical use at the time of clinical administration (See International Patent App. No. PCT/US21/63641, published as WO 2022/132982). Data were calculated for lots used in a Phase 3 clinical trial. It was calculated that pharmaceutical compositions comprising ustekinumab in accordance with embodiments herein for clinical administration comprised up to 49.78 mg acidic peak species at the time of clinical administration. It was calculated that pharmaceutical compositions comprising Reference Product for clinical administration comprised up to 24.32 mg (27.02%) acidic peak species at the time of clinical administration. It is noted that 49.78 mg acidic peak species in ustekinumab and 24.32 mg acidic peak species in Reference Product corresponds to 55.31% and 27.02%, respectively, in a 90 mg dose formulation. Patients were dosed in the Phase 3 study at 45 mg or 90 mg ustekinumab based on their weight. A maximum exposure of 49.78 mg acidic peak species corresponds to 55.31% in a 90 mg dose and 110.62% in a 45 mg dose. For clarity, 55.31% acidic peak species was present in the 90 mg dose of ustekinumab composition in accordance with embodiments herein that was administered to patients and the 110.62% was calculated based on the relative gravimetric amount of attribute exposure in the 45 mg dose. Accordingly, it is contemplated herein that pharmaceutical compositions comprising ustekinumab in accordance with embodiments herein can comprise up to 55.31% acidic peak species, or up to 100% acidic peak species. Adverse Events (AE) analyzed included occurrence of ADA (anti-drug antibody), COVID-19, nasopharyngitis, upper respiratory tract infection, hypertension, headache, proteinuria, urinary tract infection, blood pressure increase, arthralgia, skin laceration, and back pain. None of the adverse events were observed to correlate with clinical exposure to acidic peak species in the Phase 3 clinical study. Binding anti-drug antibodies were detected for 175 patients and no unusual associations were observed between binding anti-drug antibodies and acidic peak species exposure. Accordingly, it was contemplated that acidic peak species exposure can be tolerated in ustekinumab pharmaceutical compositions that remain clinically suitable. Accordingly, it is contemplated that the acidic peak species assessed herein may be tolerated in ustekinumab compositions that remain clinically suitable.

### GENERAL

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the elements described herein, in all possible variations thereof, is encompassed by the disclosure unless otherwise indicated herein or otherwise clearly contradicted by context.

1. A pharmaceutical composition comprising:
anti-interleukin 12 (IL-12)/interleukin 23 (TL-23) antibodies comprising:
a heavy chain comprising a CDRH1, CDRH2, and CDRH3, wherein the amino acid sequence of the CDRH1, CDRH2 and CDRH3 is SEQ ID NO: 1, 2 and 3, respectively; and
a light chain comprising a CDRL1, CDRL2, and CDRL3, wherein the amino acid sequence of the CDRL1, CDRL2, and CDRL3 is SEQ ID NO: 4, 5 and 6, respectively,
wherein
(a) the light chain comprises W32 (EU numbering), wherein at least 1% and no more than 2% of the light chains of the composition comprise oxidized W32; and/or
(b) the heavy chain comprises position D55 (EU numbering), and wherein greater than 6% and no more than 14.3% of said antibodies comprise isoaspartic acid at heavy chain position 55, or greater than 6% and no more than 14%, or greater than 7% and no more than 14% of said antibodies comprise isoaspartic acid at heavy chain position 55; and/or
(c) at least 0.4% and no more than 5.0%, or at least 0.4% and no more than 1.0% of said antibodies of the pharmaceutical composition are comprised by high molecular weight species selected from dimers, oligomers, and dimers and oligomers; and/or
(d) at least 1.8% and no more than 6.4%, or greater than 1.8% and no more than 6.4%, or at least 2.0% and no more than 6.4%, or at least 1.8% and no more than 3.7%, or greater than 1.8% and no more than 3.7%, or at least 2.0% and no more than 3.7% of said antibodies of the pharmaceutical composition comprise high mannose; and/or
(e) at least 26.5% and no more than 58%, or at least 26.5% and no more than 29.5%, or at least 26.5% and no more than 29%, or at least 27% and no more than 58%, or at least 27% and no more than 29.5%, or at least 27% and no more than 29% of said antibodies of the pharmaceutical composition comprise sialylation; and/or
(f) the composition comprises a total of at least 28.0%, or at least 28.0% and no more than 60.3% acidic peak species, or at least 28.0% and no more than 58.3% acidic peak species, or at least 28% and no more than 55.0% acidic peak species as determined by cation exchange high performance liquid chromatography (CEX-HPLC); and/or
(g) the heavy chain comprises W33 (EU numbered 0, wherein at least 0.1% and no more than 0.8%, or at least 0.2% and no more than 0.8%, at least 0.1% and no more than 0.4%, or at least 0.2% and no more than 0.4% of the heavy chains of the composition comprise oxidized W33.

2. The pharmaceutical composition of claim 1, wherein
(a) the light chain comprises W32 (EU numbering), wherein at least 1% and no more than 2% of the light chains of the composition comprise oxidized W32; and/or
(b) the heavy chain comprises position D55 (EU numbering), and wherein greater than 6%, and no more than 14% of said antibodies comprise isoaspartic acid at heavy chain position 55; and/or
(c) at least 0.4% and no more than 5.0% of said antibodies of the pharmaceutical composition are comprised by high molecular weight species selected from dimers, oligomers, and dimers and oligomers; and/or
(d) at least 1.8% and no more than 3.7%, or greater than 1.8% and no more than 3.7% of said antibodies of the pharmaceutical composition comprise high mannose; and/or
(e) at least 26.5% and no more than 29.5% of said antibodies of the pharmaceutical composition comprise sialylation; and/or
(f) the composition comprises a total of at least 28.0% and no more than 60.3% acidic peak species, or at least 28.0% and no more than 58.3% acidic peak species, or at least 28% and no more than 55.0% acidic peak species as determined by cation exchange high performance liquid chromatography (CEX-HPLC).

3. The pharmaceutical composition of claim 1, wherein
(a) the light chain comprises W32 (EU numbering), wherein greater than 1% and no more than 2% of the light chains of the composition comprise oxidized W32; and/or
(b) greater than 6%, and no more than 13% of said antibodies comprise isoaspartic acid at heavy chain position 55; and/or
(c) at least 0.4% and no more than 0.9%, such as at least 0.4% and no more than 0.8%, of said antibodies of the pharmaceutical composition are comprised by high molecular weight species selected from dimers, oligomers, and dimers and oligomers; and/or
(d) at least 2.0% and no more than 3.7% of said antibodies of the pharmaceutical composition comprise high mannose; and/or
(e) at least 27% and no more than 28.9% of said antibodies of the pharmaceutical composition comprise sialylation; and/or
(f) the composition comprises a total of at least 46.0% and no more than 60.3% acidic peak species, or at least 46.0% and no more than 55.0% acidic peak species, as determined by CEX-HPLC.

4. The pharmaceutical composition of claim 1, wherein
(c) greater than 0.5% and no more than 0.9%, or greater than 0.5% and no more than 0.8%, of said antibodies of the pharmaceutical composition are comprised by high molecular weight species selected from dimers, oligomers, and dimers and oligomers.

5. The pharmaceutical composition of claim 1, wherein
(a) and (b); or
(a) and (c); or
(a) and (d); or
(a) and (e); or
(a) and (f); or
(b) and (c); or
(b) and (d); or
(b) and (e); or
(b) and (f); or
(c) and (d); or
(c) and (e); or
(c) and (f); or
(d) and (e); or
(d) and (f); or
(e) and (f).

6. The pharmaceutical composition of claim 1, wherein
(a) and (b) and (c); or
(a) and (b) and (d); or
(a) and (b) and (e); or
(a) and (b) and (f); or
(a) and (c) and (d); or
(a) and (c) and (e); or
(a) and (c) and (f); or
(a) and (d) and (e); or
(a) and (d) and (f); or
(b) and (c) and (d); or
(b) and (d) and (e); or
(b) and (e) and (f); or
(c) and (d) and (e); or
(c) and (d) and (f); or
(d) and (e) and (f).

7. The pharmaceutical composition of claim 1, wherein (a), (b), (c), (d), (e), and (f).

8. The pharmaceutical composition of claim 1, wherein (a) the oxidized W32 is determined by reduced peptide mapping comprising:
denaturing one or more of the antibodies;
reducing the one or more antibodies;
S-alkylating the one or more antibodies;
digesting the one or more antibodies with trypsin, thereby producing peptides; and
subjecting said peptides to reverse phase ultra-high performance liquid chromatography (RP-UHPLC).

9. The pharmaceutical composition of claim 1, wherein (a) the oxidized W32, and/or (b) isoaspartic acid at heavy chain position 55, and/or (g) the oxidized W33, is determined by reduced peptide mapping with mass spectrometry (MS), said reduced peptide mapping comprising:
diluting one or more of the antibodies to a concentration of 1 mg/mL in 7.5M Guanidine HCl (GdnHCl), 0.25M TRIS, 2 mM EDTA, pH 7.5 and denaturing said one or more antibodies therein at 27° C. for 30 minutes;
reducing the one or more antibodies with 3 mM dithiothreitol (DTT) at 27±2° C. for 30±5 minutes;
S-alkylating the one or more antibodies with 7 mM iodoacetic acid at 27±2° C. for 15±2 minutes;
desalting the one or more antibodies;
digesting the one or more antibodies with 0.03 μg/μL trypsin at 37±2° C. for 30±5 minutes, thereby producing peptides;
quenching the digestion in 0.1 M urea; and
subjecting the peptides to RP-UHPLC on a C18 column over a gradient of 100% mobile phase A+0% mobile phase B, to 5% mobile phase A+95% mobile phase B, wherein the mobile phase gradient is generated with 0% mobile phase B from 0 min to 2 mins, 17% to 23% mobile phase B from 36 mins to 76 mins, 40% mobile phase B from 124 mins to 126 mins, 95% mobile phase B from 126.1 mins to 133 mins; and 0% mobile phase B from 133.1 mins to 144 mins, and wherein mobile phase A comprises 0.05 mM methionine 0.1% TFA in water, and wherein mobile phase B comprises 0.05 mM methionine 0.1% TFA in acetonitrile.

10. The pharmaceutical composition of claim 1, wherein (c) the percentages of high molecular weight species are as determined by size exclusion ultra-high performance liquid chromatography (SE-UHPLC).

11. The pharmaceutical composition of claim 1, wherein the (c) the percentages of high molecular weight species are as determined by SE-UHPLC comprising a 4.6×150 mm column of 1.7 μm particle size, said column equilibrated with a mobile phase of 250 mM Sodium Chloride 100 mM Sodium Phosphate pH 6.8, and detection of absorbance at 280 nm of eluate of said column, said SE-UHPLC comprising a flow rate of 0.4 mL/min, a sample temperature of 8±5° C., and 60 μg sample in an injection volume of no more than 20 μL.

12. The pharmaceutical composition of claim 1, wherein the (d) the percentages of high mannose and/or sialylation are as determined by Hydrophilic Interaction Liquid Chromatography (HILIC).

13. The pharmaceutical composition of claim 1, wherein the (d) the percentages of high mannose and/or sialylation are as determined by HILIC with in-line fluorescence detection comprising:
digesting one or more of the antibodies at a concentration of no more than 60 mg/mL in PNGase F at 37±2° C. for 120±12 minutes, thereby releasing glycans from said antibodies;
labeling said glycans with 2-aminobenzoic acid (2-AA) in a solution comprising 12.5 mg/mL 2-AA and 125 mM sodium cyanoborohydride and 37.5% (v/v) methanol; and
subjecting said labeled glycans to chromatography on a 2.1×150 mm, 1.7 μm glycan column over a gradient of 20% mobile phase A 80% mobile phase B, to 80% mobile phase A 20% mobile phase B, wherein the gradient is generated with 20% mobile phase A+80% mobile phase B from 0 to 1 mins, 42% mobile phase A+58% mobile phase B at 110 mins, 80% mobile phase A+20% mobile phase B from 111 mins to 115 mins, and 20% mobile phase A+80% mobile phase B from 120 mins to 125 mins, wherein mobile phase A comprises 100 mM ammonium formate pH 3.0, and wherein mobile phase B comprises 100% acetonitrile.

14. The pharmaceutical composition of claim 1, wherein (e) the percentages of acidic peak species are as determined by CEX-HPLC comprising:
applying the antibodies to a 4.6 mm×100 mm, 5 μm particle size cation exchange column and eluting along a salt gradient at 30° C. and pH 7.0, wherein 60 μg of the antibodies are applied to the cation exchange column in an injection volume of 60 μL;
wherein the salt gradient is generated with 5% mobile phase B from 0 min to 2 min, 5% to 12% mobile phase B from 2 min to 21 min, 12% mobile phase B from 21 min to 21.1 min, 12% to 100% mobile phase B from 21.1 min to 21.5 min; 100% mobile phase B from 21.5 min to 23.5 min, 100% to 5% mobile phase B from 23.5 min to 24 min, and to 5% mobile phase B at 24 min to 29 min,
wherein a mobile phase A comprises 20 mM sodium phosphate at pH 7.0, and the mobile phase B comprises 20 mM sodium phosphate, 500 mM sodium chloride at pH 7.0, and
wherein the mobile phase is applied to the column at a flow rate of 0.8 mL/min.

15. The pharmaceutical composition of claim 1, wherein the anti-IL-12/IL-23 antibodies inhibit IL-23 signaling as determined in an in vitro assay comprising:
(a) determination of a presence or absence of energy transfer between an amplified luminescent proximity donor bead and acceptor bead, said donor bead comprising biotinylated interleukin-12 receptor β1 (IL-12-R) immobilized thereon, and said acceptor bead comprising recombinant human interleukin-23 comprising a His tag (IL-23-His) immobilized thereon, wherein binding of said antibodies to a p40 subunit of the IL-23-His prevents the IL-23-His from binding to biotinylated IL-12-R; or
(b) determination of a presence or absence of luminescence by an engineered cell in the presence of IL-23 and said antibodies, said engineered cell expressing IL-23R, IL-12Rβ1, IL-23R, and a Sis-inducible element (SIE)-response element (RE) luciferase gene (Luc2P), wherein said SIE-RE Luc2P is responsive to IL-23 induced STAT signaling, wherein binding of said antibodies to a p40 subunit of IL-23 prevents said IL-23 from binding IL-12Rβ1.

16. The pharmaceutical composition of claim 15, wherein said anti-IL-12/IL-23 antibodies inhibit IL-23 by greater than 66%, or by at least 86% or at least 88%, compared to a reference of said IL-12/IL-23 antibodies that does not comprise oxidized light chain W32, as determined by the in vitro assay comprising (a).

17. The pharmaceutical composition of claim 15, wherein said anti-IL-12/IL-23 antibodies inhibits IL-23 to a greater degree than an ustekinumab composition wherein 44% of ustekinumab comprise oxidized light chain W32, said inhibition of IL-23 as determined by the in vitro assay comprising (a).

18. The pharmaceutical composition of claim 15, wherein said anti-IL-12/IL-23 antibodies inhibit IL-23 by greater than 54%, or by at least 89% or at least 94%, compared to a reference of said IL-12/IL-23 antibodies that does not comprise oxidized light chain W32, as determined by the in vitro assay comprising (b).

19. The pharmaceutical composition of claim 1, wherein said anti-IL-12/IL-23 antibodies comprising up to 3.7% high mannose exhibit a difference in clearance no greater than 1.6% compared to reference anti-IL-12/IL-23 antibodies comprising 0.7% high mannose, or
wherein said anti-IL-12/IL-23 antibodies comprising up to 19.3% high mannose exhibit a difference in clearance no greater than 10% compared to reference anti-IL-12/IL-23 antibodies comprising 0.7% high mannose.

20. The pharmaceutical composition of claim 1, wherein said anti-IL-12/IL-23 antibodies comprising up to 3.7% high mannose increases clearance of said antibodies by no more than 1.6%, as measured by area under curve in a pharmacokinetic model, compared to a reference anti-IL-12/IL-23 antibodies comprising 0.7% high mannose, or
wherein said anti-IL-12/IL-23 antibodies comprising up to 19.3% high mannose increases clearance of said antibodies by no more than 10%, as measured by area under curve in a pharmacokinetic model, compared to reference anti-IL-12/IL-23 antibodies comprising 0.7% high mannose.

21. The pharmaceutical composition of claim 20, wherein said anti-IL-12/IL-23 antibodies comprising up to 3.7% high mannose increases clearance of said antibodies by no more than 1.6%, as measured by area under curve in a pharmacokinetic model, compared to the reference anti-IL-12/IL-23 antibodies comprising 0.7% high mannose.

22. The pharmaceutical composition of claim 1, wherein the anti-IL-12/IL-23 antibodies have a half-life of at least 24 days.

23. The pharmaceutical composition of claim 18, wherein the half-life is as determined by quantification of free anti-IL-12/IL-23 antibodies in serum by an electrochemiluminescence (ECL) assay comprising a first anti-idiotypic antibody immobilized on a substrate and a second anti-idiotypic antibody for detection, for a dose of anti-IL-12/IL-23 antibodies of 90 mg, subcutaneous.

24. The pharmaceutical composition of claim 1, wherein the antibodies comprise the heavy chain variable region of SEQ ID NO: 7 and the light chain variable region of SEQ ID NO: 8.

25. The pharmaceutical composition of claim 1, wherein the antibodies comprise the heavy chain of SEQ ID NO: 9 and the light of SEQ ID NO: 10.

26. The pharmaceutical composition of claim 1, wherein the pharmaceutical composition is a unit dose comprising no more than 90 mg of the antibodies, such as 45 mg.

27. The pharmaceutical composition of claim 1, wherein the pharmaceutical composition is a unit dose comprising 90 mg of the antibodies.

28. The pharmaceutical composition of claim 1, wherein the pharmaceutical composition is a unit dose comprising 45 mg of the antibodies.

29-31. (canceled)

32. A method comprising administering the pharmaceutical composition of claim 1 to a subject in need of treatment by the anti-IL-12/IL-23 antibodies.

33. The method of claim 32, wherein the subject has an inflammatory condition.

34. The method of claim 32, wherein the anti-IL-12/IL-23 antibodies are administered at a dose of 45 mg, 90 mg, 130 mg, 260 mg, 390 mg, or 520 mg.

35. The method of claim 33, wherein the inflammatory condition is selected from the group consisting of psoriasis, psoriatic arthritis, and Crohn's disease.

36. A method of manufacturing the pharmaceutical composition of claim 1, comprising:
providing a composition comprising the anti-IL-12/IL-23 antibodies;
determining
an amount that light chains of the composition comprise oxidized W32;
an amount of antibodies of the pharmaceutical composition that comprise isoaspartic acid at position 55 of the heavy chain (EU numbering);
an amount of antibodies of the pharmaceutical composition that are comprised by high molecular weight species selected from dimers, oligomers, and dimers and oligomers;
an amount of antibodies of the pharmaceutical composition that comprise high mannose; or
an amount of antibodies of the pharmaceutical composition that comprise sialylation;
an amount of antibodies of the pharmaceutical composition that comprise acidic peak species; or
an amount that heavy chains of the composition comprise oxidized W33; and
manufacturing the composition for pharmaceutical use when
(a) at least 1% and no more than 2% of the light chains of the composition comprise oxidized W32;
and/or
(b) greater than 6% and no more than 14.3% of said antibodies comprise isoaspartic acid at heavy chain position 55, or greater than 6% and no more than 14%, or greater than 7% and no more than 14% of said antibodies comprise isoaspartic acid at heavy chain position 55; and/or
(c) at least 0.4% and no more than 5.0%, or at least 0.4% and no more than 1.0% of said antibodies of the pharmaceutical composition are comprised by high molecular weight species selected from dimers, oligomers, and dimers and oligomers; and/or
(d) at least 1.8% and no more than 6.4%, or greater than 1.8% and no more than 6.4%, or at least 1.8% and no more than 3.7%, or greater than 1.8% and no more than 3.7% of said antibodies of the pharmaceutical composition comprise high mannose; and/or
(e) at least 26.5% and no more than 58%, or at least 26.5% and no more than 29.5%, or at least 26.5% and no more than 29%, or at least 27% and no more than 58%, or at least 27% and no more than 29.5%, or at least 27% and no more than 29% of said antibodies of the pharmaceutical composition comprise sialylation; and/or
(f) the composition comprises a total of at least 28.0%, or at least 28.0% and no more than 60.3% acidic peak species, or at least 28.0% and no more than 58.3% acidic peak species, or at least 28% and no more than 55.0% acidic peak species as determined by CEX-HPLC; and/or
(g) at least 0.1% and no more than 0.8%, or at least 0.2% and no more than 0.8%, at least 0.1% and no more than 0.4%, or at least 0.2% and no more than 0.4% of the heavy chains of the composition comprise oxidized W33.

37. The method of claim 36, comprising manufacturing the composition for pharmaceutical use when manufacturing the composition for pharmaceutical use when
(a) at least 1% and no more than 2% of the light chains of the composition comprise oxidized W32;
and/or
(b) greater than 6%, and no more than 14%, or greater than 6%, and no more than 13% of said antibodies comprise isoaspartic acid at heavy chain position 55; and/or
(c) at least 0.4% and no more than 5.0% of said antibodies of the pharmaceutical composition are comprised by high molecular weight species selected from dimers, oligomers, and dimers and oligomers; and/or
(d) greater than 1.8% and no more than 3.7% of said antibodies of the pharmaceutical composition comprise high mannose; and/or
(e) at least 26.5% and no more than 29.5% of said antibodies of the pharmaceutical composition comprise sialylation; and/or
(f) the composition comprises a total of at least 28.0% and no more than 60.3%, or at least 28.0% and no more than 55.0% acidic peak species as determined by CEX-HPLC.

38. The method of claim 36, comprising manufacturing the composition for pharmaceutical use when (a) greater than 1% and no more than 2% of the light chains of the composition comprise oxidized W32.
